SEQUENCE LISTING (8 sequences)

SEQ ID NO: 1 - DNA, 20 bp, synthetic construct
  source 1..20: mol_type = other DNA
ggagaatgacgagtggaccc

SEQ ID NO: 2 - protein, 22 aa, synthetic construct
GSGATNFSLLKQAGDVEENPGP

SEQ ID NO: 3 - DNA, 324 bp, synthetic construct
  source 1..324: mol_type = other DNA
gacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtgg

SEQ ID NO: 4 - DNA, 357 bp, synthetic construct
  source 1..357: mol_type = other DNA
acccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccagccccctcagtgctgccacatgccatggattgcaaggacaatgtggctgacatctg

SEQ ID NO: 5 - DNA, 357 bp, synthetic construct
  source 1..357: mol_type = other DNA
acccaggatagggccaaacctgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgc

SEQ ID NO: 6 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 7 - protein, 274 aa, synthetic construct
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKR

SEQ ID NO: 8 - protein, 297 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

(19) United States
(12) Patent Application Publication
(10) Pub. No.: US 2025/0235477 A1
(43) Pub. Date: Jul. 24, 2025

(54) METHOD FOR TARGETED GENE INSERTION INTO IMMUNE CELLS

(71) Applicant: MILTENYI BIOTEC B.V. & CO. KG, Bergisch Gladbach (DE)

(72) Inventors: Dominik LOCK, Bergisch Gladbach (DE); Caroline BRANDES, Bergisch Gladbach (DE); Mario ASSENMACHER, Bergisch Gladbach (DE); Andrew KAISER, Bergisch Gladbach (DE); Stefan WILD, Bergisch Gladbach (DE)

(73) Assignee: MILTENYI BIOTEC B.V. & CO. KG, Bergisch Gladbach (DE)

(21) Appl. No.: 18/699,220

(22) PCT Filed: Sep. 28, 2022
(86) PCT No.: PCT/EP2022/077024

§ 371 Date: Apr. 5, 2024

(30) Foreign Application Priority Data
Oct. 6, 2021 (EP) 21201214.0

(51) Int. Cl. A61K 35/17; A61K 40/11; A61K 40/31; A61K 40/32; C12N 5/0783; C12N 9/22; C12N 15/11; C12N 15/86; C12N 15/90

(52) U.S. Cl. CPC A61K 35/17; A61K 40/11; A61K 40/31; A61K 40/32; C12N 5/0636; C12N 9/22; C12N 15/111; C12N 15/86; C12N 15/907; C12N 2310/20; C12N 2510/00; C12N 2750/14143

(57) ABSTRACT

The present invention provides a method for generating a composition of immune cells expressing a plurality of transgenes under the control of an endogenous promoter of said immune cells, wherein each single immune cell of said composition that underwent the process of allelic exclusion with regard to said endogenous locus expresses only one transgene wherein the transgene encodes a therapeutic protein or therapeutic nucleic acid, thereby generating a plurality of immune cells within said composition of immune cells expressing a plurality of transgenes.

### FIELD OF THE INVENTION

The invention relates to the generation of a composition of immune cells that express a plurality of transgenes under the control of an endogenous promoter of said immune cells, whereby each single immune cell of the composition expresses only one transgene, the invention relates in particular to the stable integration of said transgene into a target locus under the control of the endogenous promoter, whereby said immune cells harbor only one allele of said endogenous locus instead of the normal two alleles at an endogenous locus, e.g. due to the fact that said endogenous locus underwent allelic exclusion.

### BACKGROUND OF THE INVENTION

The use of genetically engineered immune cells such as T cells expressing a transgene such as exogenous T cell receptor (TCR) or chimeric antigen receptor (CAR) to specifically recognize and e.g. eliminate malignant cells, greatly increased the scope and potential of adoptive immunotherapy and is being assessed for new standard of care in certain human malignancies. While CARs typically target surface molecules in a human leukocyte antigen (HLA)-independent manner, TCRs allow a highly individualized therapy by targeting both tumor—as well as patient-specific processed (neo-) antigens and thereby broadening the applicability of adoptive T cell therapies. Generally, CARs comprise an extracellular antigen recognition moiety, often a single-chain variable fragment (scFv) derived from antibodies or a Fab fragment, linked to an extracellular spacer, a transmembrane domain and intracellular co-stimulatory and signaling domains. TCRs consist of two distinct chains, alpha and beta recognizing processed antigens in a HLA-dependent manner and thereby underlie MHC restriction. Thus, a specific TCR-modified T cell can only bind a processed antigen if it gets presented by a particular HLA isotype. Although TCRs represent a powerful tool for clinical applications, their application is often hampered by a low expression as well as the potential risk of exogenous alpha and/or beta chain mispairing with endogenous chains resulting in an altered specificity that might cause autoreactivity or graft-versus-host disease. Therapies using TCR- and CAR-engineered T cells, although efficacious, have a high potential for improvements, not only with regard to safety and efficacy but also in the context of manufacturing (Mock et al., Lock et al., Priesner et al.) (1-3). WO2014/184741 discloses the use of specific endonucleases including e.g. TALENs to inactivate both genes encoding the TCR as well as at least one immune checkpoint gene aiming to generate non-alloreactive T cells with an enhances cytolytic potential supposed to be used for immunotherapy.

WO2016/069282A1 discloses a method for the generation of an allogenic T cell product by combining altered gene expression of TCR alpha chain, TCR beta chain, beta-2 microglobulin, a HLA molecule, CTLA-4, PD1 and/or FAS with a transgenic expression of a TCR or CAR. WO2017/180989A2 discloses a method combining knockout of one or more endogenous genes subsequently followed by knockin of transgenes driven under the control of the endogenous promotor, respectively in T cells. Each knockout is restricted for the integration of one specific therapeutic protein.

WO2018/073391A1 discloses a method for the generation of genetically-engineered immune cells such as T cells combining site-directed gene-editing and gene insertion. Here, exogenous coding sequences are more particularly inserted under the control of endogenous promotors that are sensitive to immune cell activation. In addition, cis-acting elements downstream the coding sequence were described as possibility to insert a transgene but maintaining the expression of the endogenous gene.

WO2018/073393A2 discloses the use of TALENs aiming to generate TCRalpha/beta-negative T cells that co-express a CAR and a selection marker under the control of the TRAC promotor. Here, the selection marker allows to enrich CAR-positive T cells.

WO2020/186219A1 discloses a pooled knockin screening method to study the effect of heterologous (and/or homozygous) knockins driven under the control of an endogenous promotor e.g. TRAC in T cells.

Allelic exclusion is a process by which only one allele of a gene is expressed in a cell while the other allele is silenced.

There is a need in the art for an improved or alternative method of the generation of a composition of genetically engineered immune cells such as T cells, e.g. for use in immunotherapy of a subject suffering from a disease such as e.g. cancer.

### BRIEF DESCRIPTION OF THE INVENTION

The inventors surprisingly found a method for generating a composition (a population) of immune cells expressing at least two transgenes under the control of an endogenous promoter of said immune cells, wherein each single immune cell of said composition expresses only one transgene, wherein the transgene encodes a therapeutic protein or therapeutic nucleic acid. Two features ensure the effect of expressing only one transgene in one single cell, independent of the fact that another single cell of the composition may express another, different single transgene: first, using a modification in a homologous sequence that a) allows the integration of a transgene into a target locus of an immune cell as disclosed herein, and second, using a endogenous locus of the immune cell that e.g. underwent the process of allelic exclusion as the target locus for insertion of the transgene. Using such a locus results in the expression of only one nucleic acid sequence encoding the transgene into the immune cell, instead of two nucleic acid sequences encoding the same or two different transgenes, when the target locus is a locus that did not undergo an allelic exclusion as a locus is normally present on both alleles before an allelic exclusion may occur. Using a modification in a homologous sequence that a) allows the integration of a transgene into a target locus of an immune cell, and b) prevents the binding of the nuclease such as TALEN to said homologous sequence harboring said modification leads to a stable insertion of the transgene into the target locus also in the presence of the nuclease that normally cut out again in an equilibrium state the transgene. This has the benefit of higher numbers of cells that have integrated the transgene in a composition as compared to methods known in the art. In addition, this concept allows for simultaneous modification of immune cells with different transgenes, wherein each individual immune cell of the population has inserted only one transgene in the target locus, but wherein all transgenes in the different cells of the population generated by the methods disclosed herein are under the same target locus. This multiplexing has the benefit of reducing time and material needed for the process of several independent transduction processes as it can be performed in one single process. In addition, less immune cells as starting material are required to manufacture a clinically relevant pharmaceutical composition compared to methods known in the art.

A prominent example of the present invention may be the generation of a composition comprising T cells that express only 1 kind of TCR in a single cell but may express a plurality of different TCRs in said composition. Using the methods as disclosed herein have the effect that the transgene, e.g. comprising the T cell receptor alpha chain and the T cell receptor beta chain comprising a variable and a constant domain, respectively, will not interfere with the endogenous components of the T cell receptor alpha chain and T cell receptor beta chain as they have been knocked out. In addition, there is no interference with other variants of the T cell receptor alpha chain and the cell receptor beta chain in said immune cell although these other variants have been used in a multiplex assay of generation of the composition of immune cells as only one nucleic acid sequence can be integrated into the locus of one cell and subsequently expressed as disclosed herein. This leads to single T cells expressing purely an exogenous TCR, whereas the composition may comprise different kinds of T cells expressing purely different transgenic TCRs.

The present invention provides the methods for generating compositions comprising modified immune cells as disclosed herein and compositions obtained by said methods.

### BRIEF DESCRIPTION OF THE DRAWINGS

FIG. 1A: Schematic representation of the germline organization of the human T cell receptor beta locus with separate variable (V), diversity (D), joining (J) gene segments, and constant (C) genes on chromosome 7. During T cell development in the thymus Vβ, Dβ, and Jβ are re-arranged resulting in a functional VDJβ V-region exon that is transcribed and spliced to join to Cβ. A further nucleic acid sequence encoding a transgene, flanked with a left homology arm (LHA) and a right homology arm (RHA), integrates into the endogenous constant domain Cβ after an engineered nuclease induced a cleavage in this locus resulting in an endogenous promotor driven expression of the transgene.

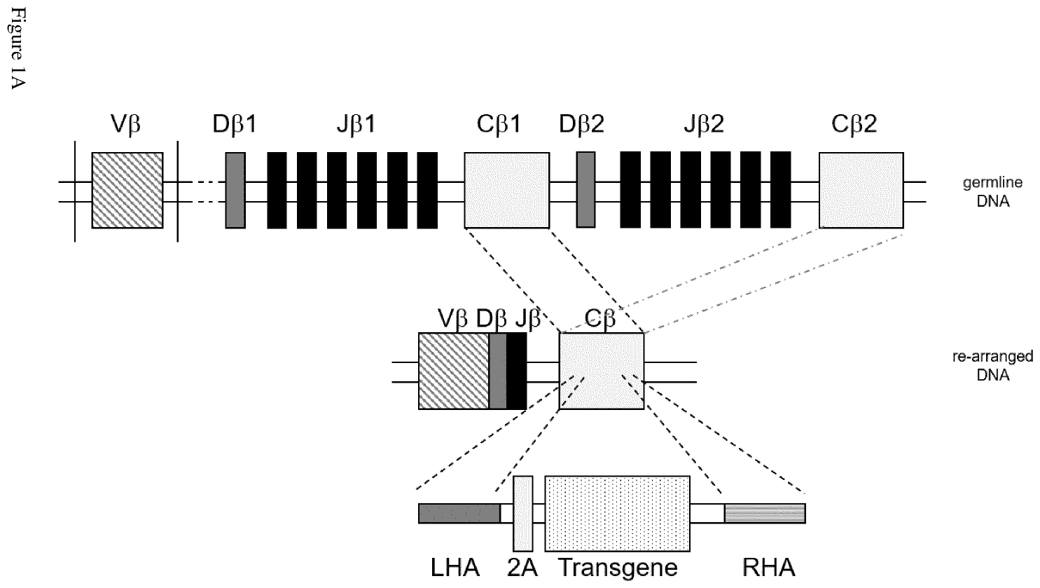

FIG. 1B: A further nucleic acid sequence encoding a transgene is flanked with a left homology arm (LHA) derived from e.g. common sequences of Exon1 of the constant domains (either TRBC1 or TRBC2) and two different right homology arms (RHA1 and RHA2). RHA1 has a nucleic acid sequence homologous to the 3′ region downstream of said cleavage site of the T cell receptor beta 1 constant gene and RHA2 has a nucleic acid sequence homologous to the 3′ region downstream of said cleavage site of the T cell receptor beta 2 constant gene. Consequently, an integration and thus expression of a transgene under the control of an endogenous promotor is possible independent of a rearrangement of Cβ1 or Cβ2.

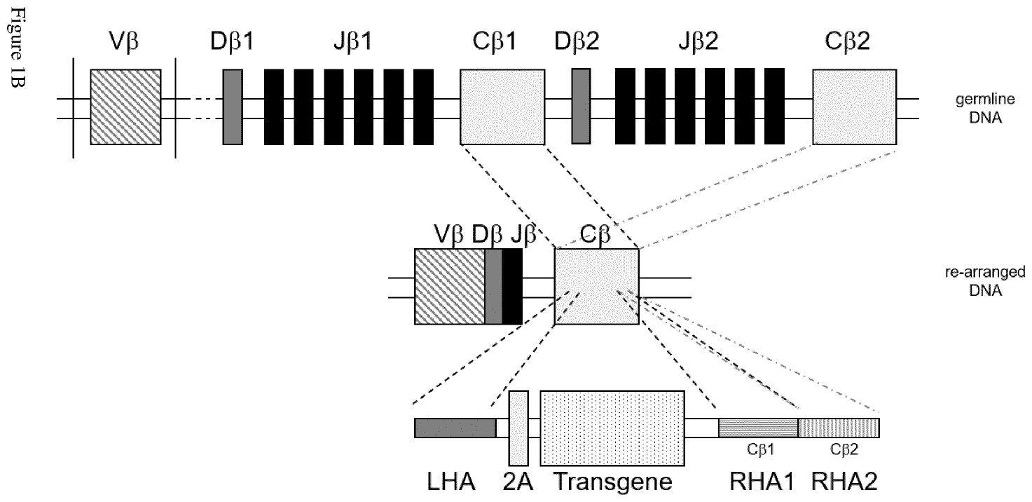

FIG. 1C: A further nucleic acid sequence encoding a transgene is flanked with two different left homology arms (LHA1 and LHA2) and two different right homology arms (RHA1 and RHA2). LHA1 has a nucleic acid sequence homologous to the 5′ region upstream of said cleavage site of the T cell receptor beta 1 constant gene and LHA2 has a nucleic acid sequence homologous to the 5′ region upstream of said cleavage site of the T cell receptor beta 2 constant gene. RHA1 has a nucleic acid sequence homologous to the 3′ region downstream of said cleavage site of the T cell receptor beta 1 constant gene and RHA2 has a nucleic acid sequence homologous to the 3′ region downstream of said cleavage site of the T cell receptor beta 2 constant gene. Alternatively, a further nucleic acid sequence encoding a transgene may be flanked with two different left homology arms (LHA1 and LHA2) and one right homology arm (RHA1).

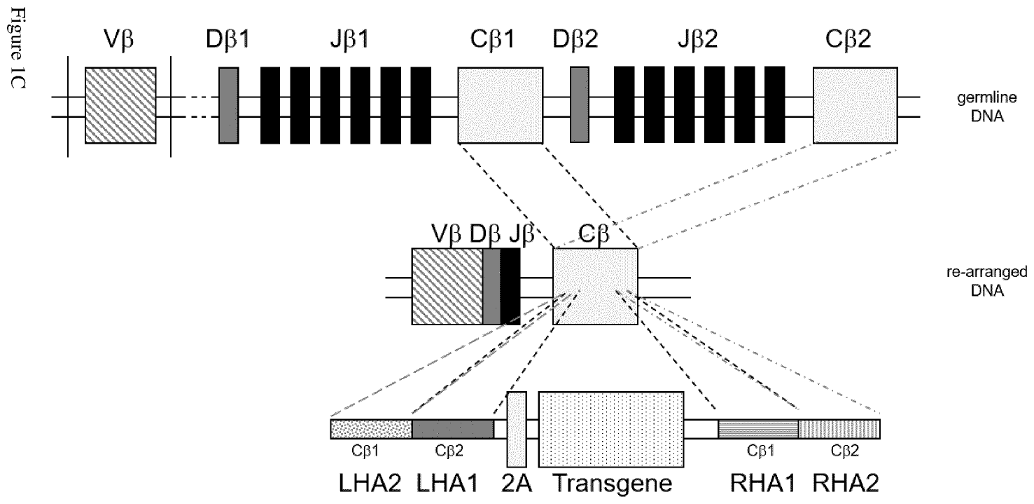

FIG. 2: Schematic workflow describing the method for generating a composition of immune cells expressing a plurality of transgenes under the control of an endogenous promoter of an endogenous locus of said immune cells wherein each single immune cell of said composition expresses only one transgene. Primary T cells expressing their endogenous TCRs (A). Introducing an engineered nuclease into the primary T cells results in an inhibition of expression of the endogenous T cell receptor on the surface of the T cell (B). Insertion of a plurality of nucleic acid sequences encoding different transgenes, e.g. TCR1, TCR2, TCR3 and TCR4, flanked with 5′ and 3′ homology arms that integrate into nuclease-cleaved site due to homologous recombination (C). Since the endogenous target locus (e.g. T cell receptor beta locus) underwent the process of allelic exclusion in T cells only one transcriptionally active and expressed allele of the originally gene is present leading to a specific expression of only one exogenous TCR, driven under the control of an endogenous promotor, per T cell (D).

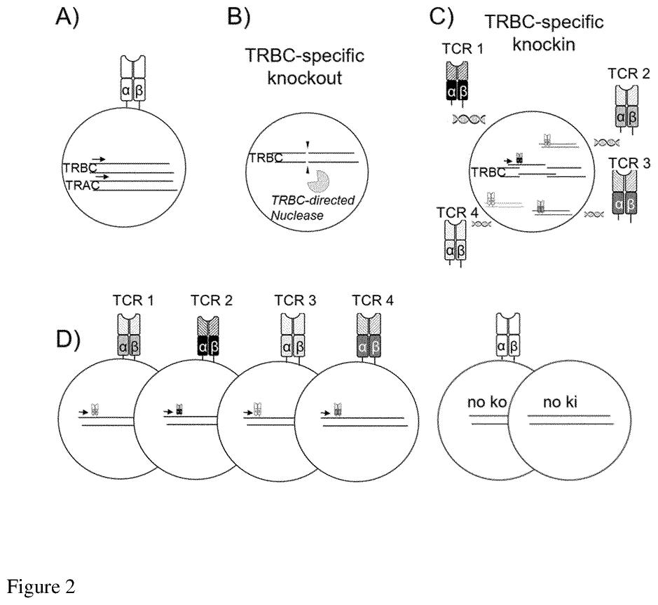

FIG. 3: Efficient knockout of TCRa/b (A), TRBC1 (B) and TRBC2 (C) expression using TRBC-specific Cas9 RNP electroporation (A). Non-transduced (mock, knockout (k.o.) only) T cells, T cells single-transduced with AAV6 containing HDR templates A, B or C, T cells double-transduced with AAV6 containing HDR templates A and B (A-B), A and C (A-C) or B and C (B-C) and T cells triple-transduced with AAV6 containing HDR templates A, B and C (A-B-C) were electroporated either without (mock) or with Cas9 complexed with TRBC-specific guide RNA at d3. TCRa/b surface expression was analyzed via staining and flow cytometry analysis at day 6. TRBC1 and TRBC2 surface expression was assessed via staining of TCRa/b and TRBC1 via flow cytometry analysis at day 22. Knockout efficiencies were calculated by normalization to TCRa/b, TRBC1 or TRBC2 expression of mock T cells.

FIG. 4: Targeted knockin of transgenes into TRBC locus through combined AAV-mediated HDR template delivery and TRBC-specific Cas9 RNP electroporation. Non-transduced (mock, knockout (k.o.) only) T cells and T cells single-transduced with HDR templates containing AAV6-TRBC-A, AAV6-TRBC-B or AAV6-TRBC-C were electroporated either without (mock) or with Cas9 complexed with TRBC-specific guide RNA at d3. Transgene expression in the TCRa/b negative T cell population (A) and CD4/CD8 T cell subsets (B) of two donors (#A, #B) was measured at d13 via staining and flow cytometry analysis. Example of an HDR template specific integration at the Cas9 cut site on genomic level. The TRBC-specific guide RNA sequence and chromatograms of the sequenced TRBC1 genomic region adjacent to the Cas9 cut site of Cas9-electroporated and AAV6-TRBC-B-transduced T cells are shown (C).

FIG. 5: Targeted knockin of transgenes into TRBC locus through combined duplexed AAV-mediated HDR template delivery and TRBC-specific Cas9 RNP electroporation. Non-transduced (mock, knockout (k.o.) only) T cells and T cells double-transduced with AAV6 containing transgene A and B (A), A and C (B) or B and C (C) were electroporated either without (mock) or with Cas9 complexed with TRBC-specific guide RNA at d3. Respective transgene expression in TCRa/b negative T cells and the fraction of transgene double positive T cells of two donors (#A, #B) was measured at d13 via staining and flow cytometry analysis.

FIG. 6: Targeted knockin of transgenes into TRBC locus through combined multiplexed AAV-mediated HDR template delivery and TRBC-specific Cas9 RNP electroporation. Non-transduced (mock, knockout (k.o.) only) T cells and T cells triple-transduced with AAV6 containing transgene A, B and C were electroporated either without (mock) or with Cas9 complexed with TRBC-specific guide RNA at d3. Respective transgene expression in TCRa/b negative T cells (A) and the fraction of transgene double positive T cells (B) of two donors (#A, #B) was measured at d13 via staining and flow cytometry analysis.

FIG. 7: Stable viability (A), cellular expansion (B) and knockout efficiency (C) of gene-edited T cells over time. Viability and cell count of non-transduced (mock, knockout (k.o.) only), single-transduced with AAV6 containing HDR template A, B or C, double-transduced with AAV6 containing HDR templates A and B (A-B), A and C (A-C) or B and C (B-C) and triple-transduced with AAV6 containing HDR templates A, B and C (A-B-C) T cells was measured using 7-AAD staining and flow cytometry analysis at day 6, day 13 and day 22. For knockout efficiency, cells were stained with TCRa/b-antibody and TCRa/b surface expression was assessed using flow cytometry at day 6, day 13 and day 22. As an example for stable knockin and transgene expression over time, GFP expression of gene-edited T cells at day 6, day 13 and day 22 measured with flow cytometry is shown (D).

### DETAILED DESCRIPTION OF THE INVENTION

In a first aspect the present invention provides a method for generating a composition (or population) of immune cells expressing a plurality of transgenes under the control of an endogenous promoter of an endogenous locus of said immune cells wherein each single immune cell of said composition (or population) expresses only one transgene, wherein the transgene encodes a therapeutic protein or therapeutic nucleic acid, the method comprising
i) introducing into primary immune cells a first nucleic acid sequence encoding an engineered nuclease, wherein said engineered nuclease induces a cleavage at a specific cleavage site within an endogenous locus of the genome of said immune cell, said cleavage resulting in an inhibition of expression of the endogenous gene of said endogenous locus,
ii) introducing into said primary immune cells a plurality of further nucleic acid sequences encoding a plurality of transgenes, wherein each individual further nucleic acid sequence of said plurality of further nucleic acid sequences comprises from 5 to 3:
a) a nucleic acid sequence homologous to the 5′ region upstream of said cleavage site (left homology arm, LHA)
b) a nucleic acid sequence encoding one transgene, and
c) a nucleic acid sequence homologous to the 3′ region downstream of said cleavage site (right homology arm, RHA),
thereby inserting (integrating) said one transgene into said endogenous locus by homologous recombination,
wherein either said LHA or said RHA is modified in such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells and thereby inserting (integrating) stably said one transgene into one endogenous locus driven under the control of an endogenous promotor in one immune cell of the composition (the population), and thereby generating a plurality of immune cells within said composition (or population) of immune cells expressing a plurality of transgenes, wherein the plurality of transgenes is at least two (different) transgenes, wherein said endogenous locus of said immune cells underwent (the process of) allelic exclusion in said immune cells, (resulting in only one transcriptionally active and expressed allele of the originally gene located in said endogenous locus).

Said primary immune cells may be a sample comprising or consisting of immune cells. Said sample comprising or consisting of immune cells may be primary human immune cells. Said immune cells may be T cells. Said sample comprising or consisting of immune cells may be obtained from a subject. Said subject may be the same subject that receive later the modified cells as disclosed herein (e.g. autologous cells in an immunotherapy) or may be a different subject (e.g. allogenic immunotherapy).

The insertion of the plurality of further nucleic acid sequence encoding the transgenes may be, by way of example but not limitation, insertion into an exon, insertion into an intron, or insertion at the 5′ end of the gene. In one embodiment, insertion of the transgene results in disruption of the endogenous gene at the site of insertion.

The modification of nucleic acid sequences of LHA or RHA may be a deletion of one or more nucleotides, an insertion of one or more nucleotides or substitution of one or more nucleotides within nucleic acid sequences of LHA of RHA. The modification may be in the binding site of the nuclease. If using a CRISPR/Cas nuclease, the modification may destroy the protospacer adjacent motif (PAM).

Said methods as disclosed herein, wherein said first nucleic acid sequence encoding an engineered nuclease may be DNA or may be RNA for a transient expression of said nuclease. Preferentially, said first nucleic acid sequence encoding an engineered nuclease is RNA (for transient expression of said nuclease).

Said plurality of transgenes may be at least three different transgenes, at least four different transgenes, at least five different transgenes or at least six different transgenes.

Said plurality of transgenes may be at least two different transgenes but not more than four different transgenes, at least two different transgenes but not more than five different transgenes, or at least two different transgenes but not more than six different transgenes,

Said methods as disclosed herein, wherein step ii) may be co-performed (together with step i)) or up to 6 hours later, dependent on the method used. Using an adeno-associated virus vector such as AAV6 step ii) may be performed preferentially 2-4 hours after step i). Linearized or plasmid DNA encoding at least one transgene may be preferentially co-electroporated in step i).

Said introduction of a first nucleic acid sequence encoding an engineered nuclease and said introduction of further nucleic acid sequences encoding the transgene(s) may be performed by electroporation and/or transduction.

Said plurality of further nucleic acid sequences encoding transgenes may be promoter-less. Such a construct allows the integration of the transgenes into a site within the genome of an immune cell such that the integrated nucleic acid sequence (transgene) is under the control of an endogenous promoter.

Said methods as disclosed herein, wherein said engineered nuclease is a meganuclease, s zinc-finger nuclease (ZFN) a transcription activator-like effector nuclease (TALE-Nuclease), a CRISPR/Cas nuclease, MAD7 nuclease, CRISPR/Cpf1, Cas12-type-derived nucleases or a megaTAL nuclease.

Preferentially said nuclease is a transcription activator-like effector nuclease (TALE-Nuclease; TALEN).

Said methods as disclosed herein, wherein said endogenous locus of said immune cells may be selected from the group consisting of T cell receptor beta locus, T cell receptor gamma locus, and T cell receptor delta locus.

The T cell receptor beta locus, T cell receptor gamma locus, and T cell receptor delta locus undergo the process of allelic exclusion in an immune cell, i.e. the T cell. Therefore, it remains one allele of said locus that is transcriptionally active and is expressed in said immune cell. The T cell receptor alpha locus may lead to incomplete allelic exclusion only, and would result in a mixture of immune cells that express only one transgene and others said express two transgenes within a single immune cell of said composition, when the composition is obtained by the method as disclosed herein.

The T cell receptor beta locus comprises either the T cell receptor beta 1 constant (TRBC1) gene or the T cell receptor beta 2 constant (TRBC2) gene. During thymocyte development of the T cell either the TRBCI gene or TRBC2 gene is re-arranged to a functional T cell receptor beta gene that is transcribed and translated as part of to the T cell receptor beta subunit of the TCR of the T cell.

Said methods as disclosed herein, wherein said endogenous locus of said immune cells may be the T cell receptor beta locus, that comprises either the T cell receptor beta 1 constant (TRBC1) gene or the T cell receptor beta 2 constant (TRBC2) gene, and said immune cells may be T cells.

Said methods as disclosed herein, wherein
a) said specific cleavage site is in exon 1 of the T cell receptor beta 1 constant gene or in exon 1 of the T cell receptor beta 2 constant gene, wherein said engineered nuclease can induce said cleavage at said specific cleavage site in exon 1 of the T cell receptor beta 1 constant gene and in exon 1 of the T cell receptor beta 2 constant gene, dependent on which T cell receptor beta constant gene is present after T cell receptor gene rearrangement during thymocyte development in said immune cell as the sequence at which is cleaved is identical in both exons 1 of the T cell receptor beta constant genes,
and wherein said each individual further nucleic acid sequence of said plurality of further nucleic acid sequences comprises downstream of said RHA (RHA1) a second RHA (RHA2), wherein said RHA 1, has a nucleic acid sequence homologous to the 3′ region downstream of said cleavage site in exon 1 of the T cell receptor beta 1 constant gene, and wherein said RHA2 has a nucleic acid sequence homologous to the 3′ region downstream of said cleavage site in exon 1 of the T cell receptor beta 2 constant gene, or vice versa, and wherein said nucleic acid sequences of RHA1 and RHA2 are different, or
b) said specific cleavage site is in exon 2 of the T cell receptor beta 1 constant gene or in exon 2 of the T cell receptor beta 2 constant gene, wherein said engineered nuclease can induce said cleavage at said specific cleavage site in exon 2 of the T cell receptor beta 1 constant gene and in exon 2 of the T cell receptor beta 2 constant gene, dependent on which T cell receptor beta constant gene is present after T cell receptor gene rearrangement during thymocyte development in said immune cell as the sequence at which is cleaved is identical in both exons 2 of the T cell receptor beta constant genes,
and wherein said each individual further nucleic acid sequence of said plurality of further nucleic acid sequences comprises upstream of said LHA (LHA1) a second LHA (LHA2), wherein said LHA 1, has a nucleic acid sequence homologous to the 5′ region upstream of said cleavage site of the T cell receptor beta 1 constant gene, and wherein said LHA2 has a nucleic acid sequence homologous to the 5′ region upstream of said cleavage site of the T cell receptor beta 2 constant gene, or vice versa, wherein said nucleic acid sequences of RHA1 and RHA2 are different, thereby allowing homologous recombination in said locus in said immune cells independently therefrom if the T cell receptor beta 1 constant gene or T cell receptor beta 2 constant gene has been re-arranged in a single immune cell of said composition.

The nucleic acid sequences of LHA1, LHA2, RHA1 and/or RHA2 may comprise exonic (Exon 1) nucleic sequences and also adjacent intronic nucleic sequences that flank the Exon 1. LHA1, LHA2, RHA1 and/or RHA2 may have between 1500 and 200 nucleotides (nt), preferentially between 250 and 600 nt, more preferentially between 350 and 450 nt.

Said nucleic acid sequence encoding one transgene may also be a nucleic acid sequence encoding a complex of transgenes. Preferentially said complex of transgenes may be a heterodimeric or hetero-multimeric protein, wherein each individual transgene of said complex of transgenes is part of said heteromeric or hetero-multimeric protein. Such a heteromeric or multimeric protein may be the complex of TCT alpha and TCR beta.

Said methods as disclosed herein, wherein the transgene(s) may be exogenous T cell receptor(s) (TCRs) and/or chimeric antigen receptor(s) (CARs).

Said methods as disclosed herein, wherein the plurality of transgenes are exogenous T cell receptors (TCRs) and wherein said nucleic acid sequence encoding said one transgene of said each individual further nucleic acid sequence of said plurality of further nucleic acid sequences comprises the T cell receptor alpha chain and the T cell receptor beta chain comprising a variable and a constant domain, respectively.

The T cell receptor alpha (or beta) chain comprises the variable domain and the constant domain of the T cell receptor alpha (or beta) gene.

The TCR alpha gene locus located on chromosome 14 consists of variable segments, joining segments and one constant region. The TCR beta gene locus located on chromosome 7 consists of variable segments followed by diversity segments, joining segments and two constant regions. The TCR alpha chain is generated by VJ recombination, whereas the beta chain is generated by VDJ recombination both involving a random joining of gene segments to generate the complete TCR chain. The diversity generated by V (D) J recombination is estimated to exceed 1015 TCRs.

An alpha/beta TCR is a heterodimeric receptor expressed on T cells recognizing processed antigens in a HLA-restricted manner. The TCR alpha and beta chains (endogenous as well as exogenous) form heterodimers that require association with the endogenous CD3 gamma, delta, epsilon, and zeta chains before they can be expressed as functional receptor on the cell surface of T cells.

Said nucleic acid sequence encoding said transgene of said each individual further nucleic acid sequence of said plurality of further nucleic acid sequences may comprise the T cell receptor alpha chain and the T cell receptor beta chain comprising a variable and a constant domain, respectively, and may also comprise a 2A element between the T cell receptor alpha chain and the T cell receptor beta chain.

Said methods as disclosed herein, wherein said transgenes may be at least two exogenous TCRs. Said at least two TCRs may be selected from the group consisting of a TCR that recognizes antigens presented by HLA A*0101, HLA A*0201, HLA A*0301,HLA A*1101, HLA A*2402, HLA A*2601, HLA A*2902, HLA A*3303, HLA A*6801, HLA B*0702, HLA B*0801, HLA B*1402, HLA B*1501, HLA B*1502, HLA B*1801, HLA B*2705, HLA B*3501, HLA B*4001, HLA B*4002, HLA B*4402**, HLA B*4403, HLA B*4501, HLA C*0102, HLA C*0202, HLA C*0303, HLA C*0304, HLA C*0401, HLA C*0501, HLA C*0602, HLA C*0701, HLA C*0702, HLA C*0801, HLA DRB1*0101, HLA DRB1*0301, HLA DRB1*0401, HLA DRB1*0701, HLA DRB1*0901, HLA DRB1*1501, HLA DRB1*1101, HLA DRB1*1301, HLA DRB1*1401, HLA DRB1*0404, HLA DRB1*0405, HLA DRB1*0407, HLA DRB1*0411, HLA DRB1*0803 and HLA DRB1*1302.

Said methods as disclosed herein, wherein said at least one further nucleic acid sequence may be a plasmid, a linearized plasmid, or a viral vector.

Said methods as disclosed herein, wherein said viral vector may be an adeno-associated virus vector such as adeno-associated virus type 6 vector (AAV6).

Said methods, wherein said transgenes may be therapeutic proteins such as a chimeric antigen receptor CARs, T cell receptors (TCR) or cytokines.

Preferentially said transgenes may be CARs and/or TCRs.

Said methods, wherein said transgenes may be therapeutic nucleic acids such as therapeutic RNAs.

In another aspect, the present invention provides a method for generating a composition of immune cells expressing a plurality of transgenes under the control of an endogenous promoter of an endogenous locus of said immune cells wherein each single immune cell of said composition that underwent (the process of) allelic exclusion with regard to said endogenous locus expresses only one transgene, wherein the transgene encodes a therapeutic protein or therapeutic nucleic acid, the method comprising
i) introducing into primary immune cells a first nucleic acid sequence encoding an engineered nuclease, wherein said engineered nuclease induces a cleavage at a specific cleavage site within an endogenous locus of the genome of said immune cell, said cleavage resulting in an inhibition of expression of the endogenous gene of said endogenous locus,
ii) introducing into said primary immune cells a plurality of further nucleic acid sequences encoding a plurality of transgenes, wherein each individual further nucleic acid sequence of said plurality of further nucleic acid sequences comprises from 5 to 3:
a) a nucleic acid sequence homologous to the 5′ region upstream of said cleavage site (left homology arm, LHA)
b) a nucleic acid sequence encoding one transgene, and
c) a nucleic acid sequence homologous to the 3′ region downstream of said cleavage site (right homology arm, RHA),
thereby inserting said one transgene into said endogenous locus by homologous recombination, wherein either said LHA or said RHA is modified in such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells and thereby inserting stably said one transgene into one endogenous locus driven under the control of an endogenous promotor in one immune cell of the composition, and thereby generating a plurality of immune cells within said composition of immune cells expressing a plurality of transgenes, wherein the plurality of transgenes is at least two different transgenes, and wherein said immune cells are T cells.

Said method, wherein said endogenous locus of said immune cells may be selected from the group consisting of T cell receptor beta locus, T cell receptor gamma locus, and T cell receptor delta locus.

Said method, wherein said endogenous locus of said immune cells may be the T cell receptor beta locus, that comprises either the T cell receptor beta 1 constant (TRBC1) gene or the T cell receptor beta 2 constant (TRBC2) gene.

In another aspect the present invention provides a method for generating a composition of immune cells expressing a plurality of transgenes under the control of an endogenous promoter of an endogenous locus of said immune cells wherein at least 80%, at least 85%, at least 90%, or at least 95% of the immune cells of said composition express only one transgene, wherein the transgene encodes a therapeutic protein or therapeutic nucleic acid, the method comprising
i) introducing into primary immune cells a first nucleic acid sequence encoding an engineered nuclease, wherein said engineered nuclease induces a cleavage at a specific cleavage site within an endogenous locus of the genome of said immune cell, said cleavage resulting in an inhibition of expression of the endogenous gene of said endogenous locus,
ii) introducing into said primary immune cells a plurality of further nucleic acid sequences encoding a plurality of transgenes, wherein each individual further nucleic acid sequence of said plurality of further nucleic acid sequences comprises from 5 to 3:
a) a nucleic acid sequence homologous to the 5′ region upstream of said cleavage site (left homology arm, LHA)
b) a nucleic acid sequence encoding one transgene, and
c) a nucleic acid sequence homologous to the 3′ region downstream of said cleavage site (right homology arm, RHA),
thereby inserting said one transgene into said endogenous locus by homologous recombination, wherein either said LHA or said RHA is modified in such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells and thereby inserting stably said one transgene into one endogenous locus driven under the control of an endogenous promotor in one immune cell of the composition, and thereby generating a plurality of immune cells within said composition of immune cells expressing a plurality of transgenes, wherein the plurality of transgenes is at least two different transgenes, wherein said endogenous locus of said immune cells underwent the process of allelic exclusion in said immune cells,

Said method, wherein said endogenous locus of said immune cells is selected from the group consisting of T cell receptor beta locus, T cell receptor gamma locus, and T cell receptor delta locus, and wherein said immune cells are T cells.

Said method, wherein said endogenous locus of said immune cells may be the T cell receptor beta locus, that comprises either the T cell receptor beta 1 constant (TRBC1) gene or the T cell receptor beta 2 constant (TRBC2) gene.

In another aspect the present invention provides a method for generating a composition of immune cells expressing a plurality of transgenes under the control of an endogenous promoter of an endogenous locus of said immune cells wherein at least 80%, at least 85%, at least 90%, or at least 95% of the immune cells of said composition express only one transgene, wherein the transgene encodes a therapeutic protein or therapeutic nucleic acid, the method comprising
i) introducing into primary immune cells a first nucleic acid sequence encoding an engineered nuclease, wherein said engineered nuclease induces a cleavage at a specific cleavage site within an endogenous locus of the genome of said immune cell, said cleavage resulting in an inhibition of expression of the endogenous gene of said endogenous locus,
ii) introducing into said primary immune cells a plurality of further nucleic acid sequences encoding a plurality of transgenes, wherein each individual further nucleic acid sequence of said plurality of further nucleic acid sequences comprises from 5 to 3:
a) a nucleic acid sequence homologous to the 5′ region upstream of said cleavage site (left homology arm, LHA)
b) a nucleic acid sequence encoding one transgene, and
c) a nucleic acid sequence homologous to the 3′ region downstream of said cleavage site (right homology arm, RHA),
thereby inserting said one transgene into said endogenous locus by homologous recombination, wherein either said LHA or said RHA is modified in such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells and thereby inserting stably said one transgene into one endogenous locus driven under the control of an endogenous promotor in one immune cell of the composition, and thereby generating a plurality of immune cells within said composition of immune cells expressing a plurality of transgenes, wherein the plurality of transgenes is at least two different transgenes, wherein said endogenous locus of said immune cells is selected from the group consisting of T cell receptor beta locus, T cell receptor gamma locus, and T cell receptor delta locus, and wherein said immune cells are T cells.

In a further aspect the present invention provides a method for generating a composition (or population) of immune cells expressing a plurality of transgenes under the control of an endogenous promoter of an endogenous locus of said immune cells wherein each single immune cell of said composition (or population) (that underwent (the process of) allelic exclusion) expresses only one transgene, wherein the transgene encodes a therapeutic protein or therapeutic nucleic acid, the method comprising
A) preparation of primary immune cells, wherein said primary immune cells are T cells,
B) magnetic separation of said T cells
C) activation of the enriched T cells using modulatory agents,
D) genetic modification of the activated T cells, wherein the genetic modification comprises the steps:
i) introducing into primary immune cells a first nucleic acid sequence encoding an engineered nuclease, wherein said engineered nuclease induces a cleavage at a specific cleavage site within an endogenous locus of the genome of said immune cell, said cleavage resulting in an inhibition of expression of the endogenous gene of said endogenous locus,
ii) introducing into said primary immune cells a plurality of further nucleic acid sequences encoding a plurality of transgenes, wherein each individual further nucleic acid sequence of said plurality of further nucleic acid sequences comprises from 5 to 3:
a) a nucleic acid sequence homologous to the 5′ region upstream of said cleavage site (left homology arm, LHA)
b) a nucleic acid sequence encoding one transgene, and
c) a nucleic acid sequence homologous to the 3′ region downstream of said cleavage site (right homology arm, RHA),
thereby inserting said one transgene into said endogenous locus by homologous recombination, wherein either said LHA or said RHA is modified in such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells and thereby inserting stably said one transgene into one endogenous locus driven under the control of an endogenous promotor in one immune cell of the composition (the population), and thereby generating a plurality of immune cells within said composition (or population) of immune cells expressing a plurality of transgenes, wherein the plurality of transgenes is at least two (different) transgenes, wherein said endogenous locus of said immune cells underwent (the process of) allelic exclusion in said immune cells, resulting in only one transcriptionally active and expressed allele of the originally gene located in said endogenous locus; and optionally
E) expansion of the genetically modified T cells,
wherein in step i) the introduction is performed by using electroporation or transduction, and
wherein in step ii) the introduction is performed by using electroporation or transduction.

In a further aspect the present invention provides a method for generating a composition (or population) of immune cells expressing a plurality of transgenes under the control of an endogenous promoter of an endogenous locus of said immune cells wherein at least 80%, at least 85%, at least 90%, or at least 95% of the immune cells of said composition express only one transgene, wherein the transgene encodes a therapeutic protein or therapeutic nucleic acid, the method comprising
A) preparation of primary immune cells, wherein said primary immune cells are T cells,
B) magnetic separation of said T cells
C) activation of the enriched T cells using modulatory agents,
D) genetic modification of the activated T cells, wherein the genetic modification comprises the steps:
i) introducing into primary immune cells a first nucleic acid sequence encoding an engineered nuclease, wherein said engineered nuclease induces a cleavage at a specific cleavage site within an endogenous locus of the genome of said immune cell, said cleavage resulting in an inhibition of expression of the endogenous gene of said endogenous locus,
ii) introducing into said primary immune cells a plurality of further nucleic acid sequences encoding a plurality of transgenes, wherein each individual further nucleic acid sequence of said plurality of further nucleic acid sequences comprises from 5′ to 3′:
a) a nucleic acid sequence homologous to the 5′ region upstream of said cleavage site (left homology arm, LHA)
b) a nucleic acid sequence encoding one transgene, and
c) a nucleic acid sequence homologous to the 3′ region downstream of said cleavage site (right homology arm, RHA),
thereby inserting said one transgene into said endogenous locus by homologous recombination, wherein either said LHA or said RHA is modified in such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells and thereby inserting stably said one transgene into one endogenous locus driven under the control of an endogenous promotor in one immune cell of the composition (the population), and thereby generating a plurality of immune cells within said composition (or population) of immune cells expressing a plurality of transgenes, wherein the plurality of transgenes is at least two (different) transgenes,
wherein said endogenous locus of said immune cells is selected from the group consisting of T cell receptor beta locus, T cell receptor gamma locus, and T cell receptor delta locus, and wherein said immune cells are T cells; and optionally
E) expansion of the genetically modified T cells,
wherein in step i) the introduction is performed by using electroporation or transduction, and
wherein in step ii) the introduction is performed by using electroporation or transduction.

Said methods as disclosed herein, wherein said methods may be performed in a closed system.

Said methods as disclosed herein, wherein said method may be an automated method (in a closed system).

Said methods as disclosed herein, wherein said primary T cells may be a sample comprising or consisting of about 2E8 T cells.

The sample comprising T cells may be provided (or obtained) from a subject such as a human (a sample comprising T cells provided by a subject). Said provided sample may be whole blood of a human, a leukapheresis of a subject, buffy coat, PBMC, outgrown or isolated T cells. Preparation of said sample may result in volume reduction, rebuffering, removal of serum, erythrocyte reduction, platelet removal, and/or washing.

Said methods as disclosed herein, wherein said primary T cells may be a sample comprising or consisting of about 2E8 T cells and wherein said plurality of further nucleic acid sequences encoding a plurality of transgenes may be two transgenes, three transgenes, four transgenes, five transgenes, six transgenes or more than six transgenes.

Normally a closed system for T cell transduction/electroporation such as the CliniMACS Prodigy (Miltenyi Biotec B.V. & Co. KG, Germany) for generation of modified T cell such as T cells expressing an exogenous TCR or CAR needs a starting population of at least 2E8 T cells (Alzubi et al.) (4).

Using the method as disclosed herein, simultaneously transducing 2, 3, 4 or more than 4 different exogenous TCRs and/CARs is possible starting with about 2E8 T cells. Methods in the art need to use independent runs for each different transgene each using about 2E8 T cells and subsequent pooling of the modified T cells, i.e. the target population of immune cells needs 8E8 T cells as a starting population, if 4 different transgenes may be transduced and subsequently pooled in one sample.

In addition, an additional step of centrifugation and pooling of the sample is necessary comprising a risk for contamination as the pooling of samples from different runs cannot be part of a closed system.

Such a closed system allows to operate under GMP or GMP-like conditions (“sterile”) resulting in cell compositions which are clinically applicable. Herein exemplarily the CliniMACS Prodigy® (Miltenyi Biotec B.V. & Co. KG, Germany) may be used as a closed system. This system is disclosed in WO2009/072003. But it is not intended to limit the use of the method of the present invention to the CliniMACS® Prodigy.

The CliniMACS Prodigy® System is designed to automate and standardize complete cellular product manufacturing processes. It combines CliniMACS® Separation Technology (Miltenyi Biotec B.V. & Co. KG, Germany) with a wide range of sensor-controlled, cell processing capabilities. Prominent features of the device are:
disposable CentriCult™ Chamber enabling standardized cell processing and cultivation
Cell enrichment and depletion capabilities, alone or combined with CliniMACS® Reagents (Miltenyi Biotec B.V. & Co. KG, Germany)
Cell cultivation and cell expansion capabilities thanks to temperature and controlled CO2 gas exchange.
Final product formulation in pre-defined medium and volume
the possibility to program the device using Flexible Programming Suite (FPS) and GAMP5 compatible programming language for customization of cell processing
Tailor-made tubing sets for a variety of applications

The centrifugation chamber and the cultivation chamber may be identical. The centrifugation chamber and the cultivation chamber can be used in various conditions: for example, for separation or transduction, high rotational speed (i.e. high g-forces) can be applied, whereas for example, culturing steps may be performed with slow rotation or even at idle state. In another variant of the invention, the chamber changes direction of rotation in an oscillating manner that results in a shaking of the chamber and maintenance of the cell in suspension. Accordingly, in the process of the invention, T cell stimulation, gene modifying and/or cultivation steps can be performed under steady or shaking conditions of the centrifugation or the cultivation chamber.

Said method, wherein said T cells are activated (stimulated) using said modulatory agents in less than 72 hours, preferentially in less than 48 hours, more preferentially in less than 24 hours, i.e. the addition of said modulatory agents and the removal of said modulatory agents occur within the period of said hours.

Said method, wherein the T cells of the provided sample may be enriched prior to said genetic modification of the T cells for CD4 positive and/or CD8 positive T cells by using CD4 and/or CD8 as positive selection marker.

In case of need, said genetically engineered T cells of step E) may be expanded to therapeutically effective amounts of cells before use in immunotherapy.

In another aspect the present invention provides a composition (a population) of immune cells expressing a plurality of transgenes under the control of an endogenous promoter of an endogenous locus of said immune cells wherein each single immune cell of said composition (or population) expresses only one transgene, wherein the transgene encodes a therapeutic protein or therapeutic nucleic acid, wherein the plurality of transgenes is at least two (different) transgenes, wherein said composition may be obtained by the methods as disclosed herein.

Said composition of immune cells, wherein at least 30%, at least 40%, at least 50%, at least 60%, at least 70%, at least 80%, at least 90% of the immune cells of said composition have stably integrated said at least one transgene under the control of said endogenous promoter.

Integration of a transgene under the control of an endogenous promoter of the immune cells using standard processes of the art lead to less than 45% cells of the composition that have stably integrated the transgene.

The equilibrium state of integration/excision in the presence of the nuclease is disrupted in the present invention leading to more stable integration of the transgene into the target locus without the risk of a subsequent excision of the transgene by the nuclease. Thus, a more efficient process leads to higher numbers of pharmaceutical relevant cells.

The composition as disclosed herein is superior compared to a composition of immune cells of the prior art:

A very high portion of the population of T cells harbors stably the transgene under the control of the endogenous promoter of the immune cell.

Normally, no additional enrichment step for getting higher concentration of target cells is necessary.

No steps for pooling of immune cells of different transgenes are necessary reducing the risk of contamination and cross-contaminations.

Due to using loci only that underwent allelic exclusion for the integration of the transgene, no interference with a second variant of the transgene can occur in engineered immune cell as disclosed herein.

As a consequence, the target cell population (the composition as disclosed herein) is less stressed than a similar cell population generated by the methods known in the art and thus the manufactured cells are more potent.

In a further aspect the present invention provides a composition as disclosed herein, wherein said composition is a pharmaceutical composition.

Said pharmaceutical composition may comprise optionally a pharmaceutically acceptable carrier.

Pharmaceutically acceptable carriers, diluents or excipients may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In another aspect the present invention provides a method for treating a subject suffering from a disease such as cancer, comprising administering to said subject the composition of immune cells as disclosed herein.

All definitions, characteristics and embodiments defined herein with regard to the first aspect of the invention as disclosed herein also apply mutatis mutandis in the context of the other aspects of the invention as disclosed herein.

### Definitions

Unless defined otherwise, technical and scientific terms used herein have the same meaning as commonly understood by one of ordinary skill in the art to which this invention belongs.

As used herein the term “comprising” or “comprises” is used in reference to compositions, methods, and respective component(s) thereof, that are essential to the method or composition, yet open to the inclusion of unspecified elements, whether essential or not.

The term “about 2E8 T cells (immune cells)” means 2E8+/−50% of T cells (immune cells) By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines.

In general, primary immune cells are provided from subjects (donors or patients) through a variety of methods known in the art, as for instance by leukapheresis techniques. The primary immune cells according to the present invention comprise also or can also be differentiated from stem cells, such as cord blood stem cells, progenitor cells, bone marrow stem cells, hematopoietic stem cells (HSC).

The term “expressing a transgene under the control of an endogenous promoter of an endogenous locus of an immune cell” means the that the nucleic acid sequence encoding the transgene is under the transcriptional control of an endogenous promoter present at the locus at that the nucleic acid sequence encoding the transgene is inserted by the present methods. Allelic exclusion is a process by which only one allele of a gene is expressed in a cell while the other allele is silenced.

As used herein, the term “locus” is the specific physical location of a DNA sequence (e.g. of a gene) in a genome. Such a locus can comprise a target sequence that is recognized and/or cleaved by a sequence-specific endonuclease according to the invention. A locus is a specific, fixed position on a chromosome where a particular gene or genetic marker is located.

The term “engineered nuclease” as used herein refers to an endonuclease. The term “endonuclease” refers to any wild-type or variant enzyme capable of catalyzing the hydrolysis (cleavage) of bonds between nucleic acids within a DNA or RNA molecule, preferably a DNA molecule. Endonucleases do not cleave the DNA or RNA molecule irrespective of its sequence, but recognize and cleave the DNA or RNA molecule at specific nucleic acid sequences, further referred to as “cleavage sites” or “target sequences” or “target sites”.

Engineered nucleases that induces a cleavage at a specific cleavage site of a nucleic acid sequence such as a genome of a cell are well known in the art and e.g. described in WO2018073393 as follows hereunder.

As used herein, the term “TALEN” or “TALE-nucleases” refers to an endonuclease comprising a DNA-binding domain comprising 14-20 or 16-22 TAL domain repeats fused to any portion of the Fokl nuclease domain.

TALE-nucleases, are fusion protein of a TALE binding domain with a cleavage catalytic domain. These endonucleases have been successfully applied to primary immune cells, in particular T cells from peripheral blood mononuclear cell (PBMC). Such TALE-nucleases, marketed under the name TALEN, are those currently used to simultaneously inactivate gene sequences in T cells originating from donors, in particular to produce allogeneic therapeutic T cells in which e.g. the gene encoding TCR (T-cell receptor) is disrupted. TALE-nucleases are very specific reagents because they need to bind DNA by pairs under obligatory heterodimeric form to obtain dimerization of the cleavage domain Fok-1. Left and right heterodimer members each recognizes a different nucleic sequence of about 14 to 20 bp, together spanning target sequences of 30 to 50 bp overall specificity.

Other endonuclease systems derived from homing endonucleases (ex: 1-Onul, or I-Crel), combined or not with TAL-nuclease (ex: MegaTAL) or zing-finger nucleases have also proven specificity, but to a lesser extend so far.

As used herein, the term “meganuclease” refers to an endonuclease that binds double-stranded DNA at a recognition sequence that is greater than 12 base pairs. Preferably, the recognition sequence for a meganuclease is 22 base pairs. A meganuclease can be an endonuclease that is derived from I-Crel, and can refer to an engineered variant of I-Crel that has been modified relative to natural I-Crel with respect to, for example, DNA-binding specificity, DNA cleavage activity, DNA-binding affinity, or dimerization properties. Methods for producing such modified variants of I-Crel are known in the art. A meganuclease as used herein binds to double-stranded DNA as a heterodimer or as a “single-chain meganuclease” in which a pair of DNA-binding domains are joined into a single polypeptide using a peptide linker. The term “homing endonuclease” is synonymous with the term “meganuclease.” Meganucleases are substantially non-toxic when expressed in cells, particularly in human T cells, such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit-Linker-C-terminal subunit. The two meganuclease subunits will generally be nonidentical in amino acid sequence and will recognize non-identical DNA sequences. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” can refer to an exogenous peptide sequence used to join two meganuclease subunits into a single polypeptide. A linker may have a sequence that is found in natural proteins, or may be an artificial sequence that is not found in any natural protein. A linker may be flexible and lacking in secondary structure or may have a propensity to form a specific three-dimensional structure under physiological conditions.

As used herein, the term “CRISPR/Cas” (Clustered Regularly Interspaced Short palindromic Repeats) refers to a caspase-based endonuclease comprising a caspase, such as Cas9, and a guide RNA that directs DNA cleavage of the caspase by hybridizing to a recognition site in the genomic DNA.

Other endonucleases reagents have been developed based on the components of the type II prokaryotic CRISPR (Clustered Regularly Interspaced Short palindromic Repeats) adaptive immune system of the bacteria S. pyogenes. This multi-component system referred to as RNA-guided nuclease system, involves members of Cas9 or Cpf1 endonuclease families coupled with a guide RNA molecules that have the ability to drive said nuclease to some specific genome sequences. Cpf1 is a single RNA-guided endonuclease that provides immunity in bacteria and can be adapted for genome editing in mammalian cells. Such programmable RNA-guided endonucleases are easy to produce because the cleavage specificity is determined by the sequence of the RNA guide, which can be easily designed and cheaply produced. The specificity of CRISPR/Cas9 although stands on shorter sequences than TAL-nucleases of about 10 pb, which must be located near a particular motif (PAM) in the targeted genetic sequence. As used herein, the term “megaTAL” refers to a single-chain nuclease comprising a transcription activator-like effector (TALE) DNA binding domain with an engineered, sequence-specific homing endonuclease.

The term “transgenic” or “exogeneous” may be used interchangeably and refer to a polypeptide or nucleic acid sequence that is foreign to a particular biological system, such as a (host) cell, and is not naturally present in that system. An exogeneous/transgenic polypeptide or nucleic acid sequence may be introduced to a biological system by artificial means, for example using recombinant techniques. For example, transgenic nucleic acid sequence encoding a polypeptide may be inserted into a suitable expression construct which is in turn used to transform a (host) cell to produce the polypeptide. A transgenic polypeptide or nucleic acid may be synthetic or artificial or may exist in a different biological system, such as a different species or cell type. An endogenous polypeptide or nucleic acid is native to a particular biological system, such as a (host) cell, and is naturally present in that system.

By “specific cleavage site” is intended a nucleic acid sequence that can be targeted and processed by an endonuclease according to the present invention. These terms refer to a specific DNA location, preferably a genomic location in a cell.

The term “cleavage” refers to the breakage of the covalent backbone of a nucleic acid sequence (a polynucleotide). As used herein, the cleavage may be initiated or induced by the engineered nuclease as disclosed herein

Homologous recombination is the exchange of DNA strands of similar or identical nucleotide sequence. Naturally, it can be used by a cell to direct error-free repair of double-strand DNA breaks. This endogenous mechanism of repair may be exploited to insert the exogenous nucleic acid sequences after cleavage at the specific cleavage site into the genome of the cell due to sufficient stretches of nucleotide sequence homology the cleavage part at the endogenous locus as disclosed herein.

The term “resulting in an inhibition of expression of the endogenous gene of said endogenous locus” means that upon binding and cutting of an engineered nuclease the endogenous gene is disrupted resulting in a complete inhibition of expression of novel endogenous protein(s) from said locus.

The term “T cell receptor gene rearrangement during thymocyte development” refers to a lymphocyte specific process in which a final sequence from a large number of potential segments is assembled. The TCR alpha chain gene locus located on chromosome 14 consists of variable segments, joining segments and the constant region. The TCR beta chain gene locus located on chromosome 7 consists of variable segments followed by two diversity segments, joining segments and two constant regions. The genetic recombination of TCR gene segments in somatic T cells occurs during the early stages of development in the thymus. The alpha chain is generated from VJ recombination and the beta chain is involved in VDJ recombination. TCR recombination occurs at two stages during the process of T cell development. First, the beta chain gene undergoes Dβ-Jβ rearrangement before Vβ-DJβ recombination in the double negative cells of the thymus. Rearrangement of the alpha chain gene takes place in double positive thymocytes. RAG1/2 bind to and introduce double strand breaks at recombination signal sequences (RSS), which flank all TCR gene segments. DNA repair machinery completes the recombination reaction.

The term “closed system” as used herein refers to any closed system which reduces the risk of cell culture contamination while performing culturing processes such as the introduction of new material, e.g. by transduction, and performing cell culturing steps such as proliferation, differentiation, activation, separation of cells, and/or electroporation if an in-line electroporation unit is connected. Such a system allows to operate under GMP or GMP-like conditions (“sterile”) resulting in cell compositions which are clinically applicable. Herein exemplarily the CliniMACS Prodigy® (Miltenyi Biotec B.V. & Co. KG, Germany) connected to the CliniMACS® Electroporator (Miltenyi Biotec B.V. & Co. KG, Germany) is used as a closed system. The CliniMACS Prodigy® is disclosed in WO2009/072003. But it is not intended to restrict the use of the method of the present invention to the CliniMACS Prodigy®. The process of the invention may be performed in a closed system, comprising a centrifugation chamber comprising a base plate and cover plate connected by a cylinder, pumps, valves, a magnetic cell separation column and a tubing set. The blood samples or other sources comprising T cells may be transferred to and from the tubing set by sterile docking or sterile welding. A suitable system is disclosed in WO2009/072003.

The closed system may comprise a plurality of tubing sets (TS) where cells are transferred between TS by sterile docking or sterile welding.

Different modules of the process may be performed in different functionally closed TS with transfer of the product (cells) of one module generated in the one tubing set to another tubing set by sterile means. For example, T cells can be magnetically enriched in a first tubing set (TS) TS100 by Miltenyi Biotec and the positive fraction containing enriched T cells is welded off the TS100 and welded onto a second tubing set TS730 by Miltenyi Biotec for further activation, modification, cultivation and washing.

The terms “automated method” or “automated process” as used herein refer to any process being automated through the use of devices and/or computers and computer software. Methods (processes) that have been automated require less human intervention and less human time. In some instances the method of the present invention is automated if at least one step of the present method is performed without any human support or intervention. Preferentially the method of the present invention is automated if all steps of the method as disclosed herein are performed without human support or intervention other than connecting fresh reagents to the system. Preferentially the automated process is implemented on a closed system such as CliniMACS Prodigy® as disclosed herein.

The closed system may comprise a) a sample processing unit comprising an input port and an output port coupled to a rotating container (or centrifugation chamber) having at least one sample chamber, wherein the sample processing unit is configured to provide a first processing step to a sample or to rotate the container so as to apply a centrifugal force to a sample deposited in the chamber and separate at least a first component and a second component of the deposited sample; and b) a sample separation unit coupled to the output port of the sample processing unit, the sample separation unit comprising a separation column holder, a pump, and a plurality of valves configured to at least partially control fluid flow through a fluid circuitry and a separation column positioned in the holder, wherein the separation column is configured to separate labeled and unlabeled components of sample flown through the column.

Said rotating container may also be used as a temperature controlled cell incubation and cultivation chamber (CentriCult Unit=CCU). This chamber may be flooded with defined gas mixes, provided by an attached gas mix unit (e.g. use of pressurized air/N2/CO2 or N2/CO2/O2).

All agents may be connected to the closed system before process initiation. This comprises all buffers, solutions, cultivation media and supplements, MicroBeads, used for washing, transferring, suspending, cultivating, harvesting cells or immunomagnetic cell sorting within the closed system. Alternatively, such agents might by welded or connected by sterile means at any time during the process.

The cell sample comprising T cells may be provided in transfer bags or other suited containers which can be connected to the closed system by sterile means.

The term “providing a (cell) sample comprising T cells” means the provision of a cell sample, preferentially of a human cell sample of hematologic origin. Normally, the cell sample may be composed of hematologic cells from a donor or a patient. Such blood product can be in the form of whole blood, buffy coat, leukapheresis, PBMCs or any clinical sampling of blood product. It may be from fresh or frozen origin.

The term “washing” means for example the replacement of the medium or buffer in which the cells are kept. The replacement of the supernatant can be in part (example 50% of the medium is removed and 50% fresh medium is added) this often is applied for dilution or feeding purposes, or entirely. Several washing steps may be combined in order to obtain a more profound replacement of the original medium in which the cells are kept. A washing step often may involve pelleting the cells by centrifugation forces and removing the supernatant. In the method of the present invention, cells may be pelleted by rotation of the chamber at e.g. 300×g and the supernatant may be removed during rotation of the chamber. Medium may be added during rotation or at steady state.

Generally, the washing or washing step may be performed once or by a series of media/buffer exchanges (at least twice exchanges, e.g. 2, 3, 4, 5, 6, 7, 8, 9 or 10 exchanges) thereby removing the substances intended to be removed from the T cells such as human serum and/or its components, the magnetic particles or the residual lentiviral vector particles. The exchanges may be performed by separation of cells and media/buffer by centrifugation, sedimentation, adherence or filtration and subsequent exchange of media/buffer.

The modulatory agents may be selected from the group consisting of agonistic antibodies or antigen binding fragment thereof, cytokines, recombinant costimulatory molecules and small drug inhibitors. Said modulatory agents may be anti-CD3 and anti-CD28 antibodies or antigen-binding fragments thereof coupled to beads or nanostructures. The modulatory agents may be a nanomatrix, the nanomatrix comprising a) a matrix of mobile polymer chains, and b) attached to said matrix of mobile polymer chains anti-CD3 and anti-CD28 antibodies or antigen-binding fragments thereof, wherein the nanomatrix is 1 to 500 nm in size. The anti-CD3 and anti-CD28 antibodies or antigen-binding fragments thereof may be attached to the same or to separate matrices of mobile polymer chains. If the anti-CD3 and anti-CD28 antibodies or antigen-binding fragments thereof may be attached to separate matrices of mobile polymer chains, fine-tuning of nanomatrices for the stimulation of the T cells may be possible. The nanomatrix may be biodegradable. The nanomatrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate.

The term “particle” as used herein refers to a solid phase such as colloidal particles, microspheres, nanoparticles, or beads. Methods for generation of such particles are well known in the field of the art. The particles may be magnetic particles. The particles may be in a solution or suspension or they may be in a lyophilised state prior to use in the present invention. The lyophilized particle is then reconstituted in convenient buffer before contacting the sample to be processed regarding the present invention.

The term “magnetic” in “magnetic particle” as used herein refers to all subtypes of magnetic particles which can be prepared with methods well known to the skilled person in the art, especially ferromagnetic particles, superparamagnetic particles and paramagnetic particles. “Ferromagnetic” materials are strongly susceptible to magnetic fields and are capable of retaining magnetic properties when the field is removed. “Paramagnetic” materials have only a weak magnetic susceptibility and when the field is removed quickly lose their weak magnetism. “Superparamagnetic” materials are highly magnetically susceptible, i.e. they become strongly magnetic when placed in a magnetic field, but, like paramagnetic materials, rapidly lose their magnetism.

For enrichment, isolation or selection in principle any sorting technology can be used. This includes for example affinity chromatography or any other antibody-dependent separation technique known in the art. Any ligand-dependent separation technique known in the art may be used in conjunction with both positive and negative separation techniques that rely on the physical properties of the cells. An especially potent sorting technology is magnetic cell sorting. Methods to separate cells magnetically are commercially available e.g. from Invitrogen, Stem cell Technologies, in Cellpro, Seattle or Advanced Magnetics, Boston. For example, monoclonal antibodies can be directly coupled to magnetic polystyrene particles like Dynal M 450 or similar magnetic particles and used e.g. for cell separation. The Dynabeads technology is not column based, instead these magnetic beads with attached cells enjoy liquid phase kinetics in a sample tube, and the cells are isolated by placing the tube on a magnetic rack. However, in a preferred embodiment for enriching CD4+ and/or CD8+ T cells from a sample comprising T cells according the present invention monoclonal antibodies or antigen binding fragments thereof are used in conjunction with colloidal superparamagnetic microparticles having an organic coating by e.g. polysaccharides (Magnetic-activated cell sorting (MACS) technology (Miltenyi Biotec B.V. & Co. KG, Germany)). These particles (nanobeads or MicroBeads) can be either directly conjugated to monoclonal antibodies or used in combination with anti-immunoglobulin, avidin or anti-hapten-specific MicroBeads.

The MACS technology allows cells to be separated by incubating them with magnetic nanoparticles coated with antibodies directed against a particular surface antigen. This causes the cells expressing this antigen to attach to the magnetic nanoparticles. Afterwards the cell solution is transferred on a column placed in a strong magnetic field. In this step, the cells attach to the nanoparticles (expressing the antigen) and stay on the column, while other cells (not expressing the antigen) flow through. With this method, the cells can be separated positively or negatively with respect to the particular antigen(s)/marker(s).

In case of a positive selection the cells expressing the antigen(s) of interest, which attached to the magnetic column, are washed out to a separate vessel, after removing the column from the magnetic field.

In case of a negative selection the antibody used is directed against surface antigen(s) which are known to be present on cells that are not of interest. After application of the cells/magnetic nanoparticles solution onto the column the cells expressing these antigens bind to the column and the fraction that goes through is collected, as it contains the cells of interest. As these cells are non-labelled by an antibody coupled to nanoparticels, they are “untouched”.

The procedure can be performed using direct magnetic labelling or indirect magnetic labelling. For direct labelling the specific antibody is directly coupled to the magnetic particle. Indirect labelling is a convenient alternative when direct magnetic labelling is not possible or not desired. A primary antibody, a specific monoclonal or polyclonal antibody, a combination of primary antibodies, directed against any cell surface marker can be used for this labelling strategy. The primary antibody can either be unconjugated, biotinylated, or fluorophore-conjugated. The magnetic labelling is then achieved with anti-immunoglobulin MicroBeads, anti-biotin MicroBeads, or anti-fluorophore MicroBeads.

As used herein, the term “antigen” is intended to include substances that bind to or evoke the production of one or more antibodies and may comprise, but is not limited to, proteins, peptides, polypeptides, oligopeptides, lipids, carbohydrates such as dextran, haptens and combinations thereof, for example a glycosylated protein or a glycolipid. The term “antigen” as used herein refers to a molecular entity that may be expressed on the surface of a target cell and that can be recognized by means of the adaptive immune system including but not restricted to antibodies or TCRs, or engineered molecules including but not restricted to endogenous or transgenic TCRs, CARs, scFvs or multimers thereof, Fab-fragments or multimers thereof, antibodies or multimers thereof, single chain antibodies or multimers thereof, or any other molecule that can execute binding to a structure with high affinity. The tumor associated antigen (TAA) as used herein refers to an antigenic substance produced in tumor cells. Tumor associated antigens are useful tumor or cancer markers in identifying tumor/cancer cells with diagnostic tests and are potential candidates for use in cancer therapy. Preferentially, the TAA may be expressed on the cell surface of the tumor/cancer cell, so that it may be recognized by the antigen binding receptor as disclosed herein.

The term “antigen-binding molecule” as used herein refers to any molecule that binds preferably to or is specific for the desired target molecule of the cell, i.e. the antigen. The term “antigen-binding molecule” comprises e.g. an antibody or antigen binding fragment thereof. The term “antibody” as used herein refers to polyclonal or monoclonal antibodies, which can be generated by methods well known to the person skilled in the art. The antibody may be of any species, e.g. murine, rat, sheep, human. For therapeutic purposes, if non-human antigen binding fragments are to be used, these can be humanized by any method known in the art. The antibodies may also be modified antibodies (e.g. oligomers, reduced, oxidized and labeled antibodies).

The term “antibody” comprises both intact molecules and antigen binding fragments, such as Fab, Fab′, F(ab′)2, Fv, nanobodies and single-chain antibodies. Additionally, the term “antigen-binding fragment” includes any molecule other than antibodies or antibody fragments that binds preferentially to the desired target molecule of the cell. Suitable molecules include, without limitation, oligonucleotides known as aptamers that bind to desired target molecules, carbohydrates, lectins or any other antigen binding protein (e.g. receptor-ligand interaction). The linkage (coupling) between antibody and particle or nanostructure can be covalent or non-covalent. A covalent linkage can be, e.g. the linkage to carboxyl-groups on polystyrene beads, or to NH2 or SH2 groups on modified beads. A non-covalent linkage is e.g. via biotin-avidin or a fluorophore-coupled-particle linked to anti-fluorophore antibody.

The terms “specifically binds to” or “specific for” with respect to an antigen-binding molecule, e.g. an antibody or antigen-binding fragment thereof, refer to an antigen-binding molecule (in case of an antibody or antigen-binding fragment thereof to an antigen-binding domain) which recognizes and binds to a specific antigen in a sample, e.g. CD4, but does not substantially recognize or bind other antigens in said sample. An antigen-binding domain of an antibody or antigen-binding fragment thereof that binds specifically to an antigen from one species may bind also to that antigen from another species. This cross-species reactivity is not contrary to the definition of “specific for” as used herein. An antigen-binding domain of an antibody or antigen-binding fragment thereof that specifically binds to an antigen, e.g. the CD4 antigen, may also bind substantially to different variants of said antigen (allelic variants, splice variants, isoforms etc.). This cross reactivity is not contrary to the definition of that antigen-binding domain as specific for the antigen, e.g. for CD4.

The terms “genetically modified immune cell (T cell)” or “engineered immune cell (T cell)” may be used interchangeably and mean containing and/or expressing a foreign gene or nucleic acid sequence which in turn modifies the genotype or phenotype of the cell or its progeny. Especially, the terms refer to the fact that cells can be manipulated by recombinant methods well known in the art to express stably or transiently peptides or proteins, e.g. CARs which are not expressed in these cells in the natural state. Genetic modification of cells may include but is not restricted to transfection, electroporation, nucleofection, transduction using retroviral vectors, lentiviral vectors, non-integrating retro- or lentiviral vectors, transposons, designer nucleases including zinc finger nucleases, TALENs or CRISPR/Cas.

As used herein “autologous” means that cells, a cell line, or population of cells used for treating subjects are originating from said subject.

As used herein “allogeneic” means that cells or population of cells used for treating subjects are not originating from said subject but from a donor.

The terms “immune cell” or “immune effector cell” may be used interchangeably and refer to a cell that may be part of the immune system and executes a particular effector function such as T cells, alpha-beta T cells, NK cells, NKT cells, B cells, innate lymphoid cells (ILC), cytokine induced killer (CIK) cells, lymphokine activated killer (LAK) cells, gamma-delta T cells, regulatory T cells (Treg), monocytes or macrophages. Preferentially these immune cells are human immune cells. Preferred immune cells are cells with cytotoxic effector function such as alpha-beta T cells, NK cells, NKT cells, ILC, CIK cells, LAK cells or gamma-delta T cells.

Most preferred immune effector cells are T cells and/or NK cells. Tumor infiltrating lymphocytes (TILs) are T cells that have moved from the blood of a subject into a tumor. These TILs may be removed from a patient's tumor by methods well known in the art, e.g. enzymatic and mechanic tumor disruption followed by density centrifugation and/or cell marker specific enrichment. TILs are genetically engineered as disclosed herein, and then given back to the patient. “Effector function” means a specialized function of a cell, e.g. in a T cell an effector function may be cytolytic activity or helper activity including the secretion of cytokines. T cells or T lymphocytes are a type of lymphocyte that play a central role in cell-mediated immunity. They can be distinguished from other lymphocytes, such as B cells and natural killer cells (NK cells), by the presence of a T cell receptor (TCR) on the cell surface. There are several subsets of T cells, each with a distinct function.

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, Th9, or TFH, which secrete different cytokines to facilitate a different type of immune response. Signaling from the APC directs T cells into particular subtypes.

Cytotoxic T cells (TC cells, or CTLs) destroy virally infected cells and tumor cells and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described-Foxp3+ Treg cells and Foxp3-Treg cells.

Natural killer T cells (NKT cells—not to be confused with natural killer cells of the innate immune system) bridge the adaptive immune system with the innate immune system. Unlike conventional T cells that recognize peptide antigens presented by major histocompatibility complex (MHC) molecules, NKT cells recognize glycolipid antigen presented by a molecule called CD1d. Once activated, these cells can perform functions ascribed to both Th and Tc cells (i.e., cytokine production and release of cytolytic/cell killing molecules).

The term “natural killer cells (NK cells)” are defined as large granular lymphocytes (LGL) and constitute the third kind of cells differentiated from the common lymphoid progenitor-generating B and T lymphocytes. NK cells are known to differentiate and mature in the bone marrow, lymph nodes, spleen, tonsils, and thymus, where they then enter the circulation. NK cells differ from natural killer T cells (NKTs) phenotypically, by origin and by respective effector functions; often, NKT cell activity promotes NK cell activity by secreting IFNγ. In contrast to NKT cells, NK cells do not express T cell antigen receptors (TCR) or pan T marker CD3 or surface immunoglobulins (Ig) B cell receptors, but they usually express the surface markers CD16 (FcγRIII) and CD56 in humans, NK1.1 or NK1.2 in C57BL/6 mice. Up to 80% of human NK cells also express CD8. Continuously growing NK cell lines can be established from cancer patients and common NK cell lines are for instance NK-92, NKL and YTS.

Immunotherapy is a medical term defined as the “treatment of disease by inducing, enhancing, or suppressing an immune response”. Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based, preferentially T cell-based or NK cell-based cytotoxic responses to attack cancer cells. T cells that have a natural or genetically engineered reactivity to a patient's cancer are generated in-vitro and then transferred back into the cancer patient. Then the immunotherapy is referred to as “CAR cell immunotherapy” or in case of use of T cells only as “CAR T cell therapy” or “CAR T cell immunotherapy”, when these cells express a CAR.

The term “treatment” as used herein means to reduce the frequency or severity of at least one sign or symptom of a disease.

The terms “therapeutically effective amount” or “therapeutically effective population” mean an amount of a cell population which provides a therapeutic benefit in a subject.

As used herein, the term “subject” refers to an animal. Preferentially, the subject is a mammal such as mouse, rat, cow, pig, goat, chicken dog, monkey or human. More preferentially, the subject is a human. The subject may be a subject suffering from a disease such as cancer (a patient) or from an autoimmune disease or from a allergic disease or from an infectious disease or from graft rejection.

The term “expression” as used herein is defined as the transcription and/or translation of a particular nucleotide sequence driven by its promoter in a cell.

The term “cancer” is known medically as a malignant neoplasm. Cancer is a broad group of diseases involving unregulated cell growth and includes all kinds of leukemia. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The terms “nucleic acid”, “nucleic acid sequence/molecule” or “polynucleotide” as used interchangeably herein refer to polymers of nucleotides. Polynucleotides, which can be hydrolyzed into monomeric “nucleotides.” The monomeric nucleotides can be hydrolyzed into nucleosides. As used herein, the term “polynucleotides” encompasses, but is not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

The term “transgene driven under the control of an endogenous promoter in an immune cell” means that the transgene is operably linked to an endogenous regulatory element, i.e. an endogenous promoter of said immune cell.

The term “operably linked” refers to functional linkage between a regulatory sequence and a transgenic nucleic acid sequence resulting in expression of the latter.

As used herein, the terms “promoter” or “regulatory sequence” mean a nucleic acid sequence which is required for transcription of a gene product operably linked to the promoter/regulatory sequence. The promoter/regulatory sequence may, for example, be one which expresses the gene product in a tissue-specific manner, e.g. in a T cell specific manner.

The term “activation” as used herein refers to inducing physiological changes with a cell that increase target cell function, proliferation and/or differentiation.

The term “transduction” means the transfer of genetic material from a viral agent such as a lentiviral vector particle into a eukaryotic cell such as a T cell.

The term “electroporation” is a technique in which an electrical field is applied to cells in order to increase the permeability of the cell membrane, allowing chemicals, drugs, or DNA to be introduced into the cell.

A transgene may be a gene that has been transferred by genetic engineering techniques into a host that normally does nor bear this gene. The gene may be a naturally gene that occurs in other cells or may be a recombinant gene. Most prominent transgenes used in the present invention may be the T cell receptor and the chimeric antigen receptor.

The T cell receptor (TCR) is a protein complex found on the surface of T cells, or T lymphocytes, that is responsible for recognizing fragments of antigen as peptides bound to major histocompatibility complex (MHC) molecules.

The TCR is composed of two different protein chains (that is, it is a heterodimer). In humans, in 95% of T cells the TCR consists of an alpha (α) chain and a beta (β) chain (encoded by TRA and TRB, respectively), whereas in 5% of T cells the TCR consists of gamma and delta (y/8) chains (encoded by TRG and TRD, respectively). This ratio changes during ontogeny and in diseased states (such as leukemia). Each locus can produce a variety of polypeptides with constant and variable regions.

When the TCR engages with antigenic peptide and MHC (peptide/MHC), the T lymphocyte (T cell) is activated through signal transduction, that is, a series of biochemical events mediated by associated enzymes, co-receptors, specialized adaptor molecules, and activated or released transcription factors.

Structural characteristics: The TCR is a disulfide-linked membrane-anchored heterodimeric protein normally consisting of the highly variable alpha (a) and beta (B) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells.

Each chain is composed of two extracellular domains: Variable (V) region and a Constant (C) region, both of Immunoglobulin superfamily (IgSF) domain forming antiparallel β-sheets. The Constant region is proximal to the cell membrane, followed by a transmembrane region and a short cytoplasmic tail, while the Variable region binds to the peptide/MHC complex.

The variable domain of both the TCR α-chain and β-chain each have three hypervariable or complementarity-determining regions (CDRs). There is also an additional area of hypervariability on the β-chain (HV4) that does not normally contact antigen and, therefore, is not considered a CDR.

The residues in these variable domains are located in two regions of the TCR, at the interface of the α- and β-chains and in the β-chain framework region that is thought to be in proximity to the CD3 signal-transduction complex. CDR3 is the main CDR responsible for recognizing processed antigen, although CDR1 of the alpha chain has also been shown to interact with the N-terminal part of the antigenic peptide, whereas CDR1 of the β-chain interacts with the C-terminal part of the peptide.

CDR2 is thought to recognize the MHC. CDR4 of the β-chain is not thought to participate in antigen recognition, but has been shown to interact with superantigens.

The constant domain of the TCR consists of short connecting sequences in which a cysteine residue forms disulfide bonds, which form a link between the two chains.

Generation of the TCR diversity: The generation of TCR diversity is similar to that for antibodies and B-cell antigen receptors. It arises mainly from genetic recombination of the DNA-encoded segments in individual somatic T cells by somatic V (D) J recombination using RAG1 and RAG2 recombinases. Unlike immunoglobulins, however, TCR genes do not undergo somatic hypermutation, and T cells do not express activation-induced cytidine deaminase (AID). The recombination process that creates diversity in BCR (antibodies) and TCR is unique to lymphocytes (T and B cells) during the early stages of their development in primary lymphoid organs (thymus for T cells, bone marrow for B cells).

Each recombined TCR possess unique antigen specificity, determined by the structure of the antigen-binding site formed by the α and β chains in case of αβ T cells or γ and δ chains on case of γδ T cells.

The TCR alpha chain is generated by VJ recombination, whereas the beta chain is generated by VDJ recombination (both involving a random joining of gene segments to generate the complete TCR chain).

Likewise, generation of the TCR gamma chain involves VJ recombination, whereas generation of the TCR delta chain occurs by VDJ recombination.

The intersection of these specific regions (V and J for the alpha or gamma chain; V, D, and J for the beta or delta chain) corresponds to the CDR3 region that is important for peptide/MHC recognition (see above).

It is the unique combination of the segments at this region, along with palindromic and random nucleotide additions (respectively termed “P-” and “N-”), which accounts for the even greater diversity of T cell receptor specificity for processed antigenic peptides.

Later during development, individual CDR loops of TCR can be re-edited in the periphery outside thymus by reactivation of recombinases using a process termed TCR revision (editing) and change its antigenic specificity.

The TCR complex: In the plasma membrane the TCR receptor chains α and β associate with six additional adaptor proteins to form an octameric complex. The complex contains both a and β chains, forming the ligand-binding site, and the signaling modules CD3δ, CD3γ, CD3ε and CD3ζ in the stoichiometry TCR α β-CD3εγ-CD3εδ-CD3ζζ. Charged residues in the transmembrane domain of each subunit form polar interactions allowing a correct and stable assembly of the complex.

Antigen discrimination: Each T cell expresses clonal TCRs which recognize a specific peptide loaded on a MHC molecule (pMHC), either on MHC class II on the surface of antigen-presenting cells or MHC class I on any other cell type. A unique feature of T cells is their ability to discriminate between peptides derived from healthy, endogenous cells and peptides from foreign or abnormal (e.g. infected or cancerous) cells in the body. Antigen presenting cells do not discriminate between self and foreign peptides and typically express a large number of self-derived pMHC on their cell surface and only a few copies of any foreign pMHC.

Because T cells undergo positive selection in the thymus there is a non-negligible affinity between self pMHC and the TCR, nevertheless, the T cell receptor signaling should not be activated by self pMHC such that endogenous, healthy cells are ignored by T cells. However, when these very same cells contain even minute quantities of pathogen derived pMHC, T cells must get activated and initiate immune responses.

In general, a CAR as used herein may comprise an extracellular domain (extracellular part) comprising the antigen binding domain, a transmembrane domain and a cytoplasmic signaling domain (intracellular signaling domain). The extracellular domain may be linked to the transmembrane domain by a linker or spacer. The extracellular domain may also comprise a signal peptide. In some embodiments of the invention the antigen binding domain of a CAR binds a tag or hapten that is coupled to a polypeptide (“haptenylated” or “tagged” polypeptide), wherein the polypeptide may bind to a disease-associated antigen such as a tumor associated antigen (TAA) that may be expressed on the surface of a cancer cell. Such a CAR may be referred to as “anti-tag” CAR or “adapterCAR” or “universal CAR” as disclosed e.g. in U.S. Pat. No. 9,233,125B2.

The haptens or tags may be coupled directly or indirectly to a polypeptide (the tagged polypeptide), wherein the polypeptide may bind to said disease associated antigen expressed on the (cell) surface of a target. The tag may be e.g. dextran or a hapten such as biotin or fluorescein isothiocyanate (FITC) or phycoerythrin (PE) or thiamin, but the tag may also be a peptide sequence e.g. chemically or recombinantly coupled to the polypeptide part of the tagged polypeptide. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv, respectively.

A “signal peptide” refers to a peptide sequence that directs the transport and localization of the protein within a cell, e.g. to a certain cell organelle (such as the endoplasmic reticulum) and/or the cell surface.

Generally, an “antigen binding domain” refers to the region of the CAR that specifically binds to an antigen, e.g. to a tumor associated antigen (TAA) or tumor specific antigen (TSA). The CARs of the invention may comprise one or more antigen binding domains (e.g. a tandem CAR). Generally, the targeting regions on the CAR are extracellular. The antigen binding domain may comprise an antibody or an antigen binding fragment thereof. The antigen binding domain may comprise, for example, full length heavy chain, Fab fragments, single chain Fv (scFv) fragments, divalent single chain antibodies or diabodies. Any molecule that binds specifically to a given antigen such as affibodies or ligand binding domains from naturally occurring receptors may be used as an antigen binding domain. Often the antigen binding domain is a scFv. Normally, in a scFv the variable regions of an immunoglobulin heavy chain and light chain are fused by a flexible linker to form a scFv. Such a linker may be for example the “(G₄/S)₃-linker”.

In some instances, it is beneficial for the antigen binding domain to be derived from the same species in which the CAR will be used in. For example, when it is planned to use it therapeutically in humans, it may be beneficial for the antigen binding domain of the CAR to comprise a human or humanized antibody or antigen binding fragment thereof. Human or humanized antibodies or antigen binding fragments thereof can be made by a variety of methods well known in the art.

“Spacer” or “hinge” as used herein refers to the hydrophilic region which is between the antigen binding domain and the transmembrane domain. The CARs of the invention may comprise an extracellular spacer domain but is it also possible to leave out such a spacer. The spacer may include e.g. Fc fragments of antibodies or fragments thereof, hinge regions of antibodies or fragments thereof, CH2 or CH3 regions of antibodies, accessory proteins, artificial spacer sequences or combinations thereof. A prominent example of a spacer is the CD8alpha hinge. The transmembrane domain of the CAR may be derived from any desired natural or synthetic source for such domain. When the source is natural the domain may be derived from any membrane-bound or transmembrane protein. The transmembrane domain may be derived for example from CD8alpha or CD28. When the key signaling and antigen recognition modules (domains) are on two (or even more) polypeptides then the CAR may have two (or more) transmembrane domains. The splitting key signaling and antigen recognition modules enable for a small molecule-dependent, titratable and reversible control over CAR cell expression (e.g. WO2014127261A1) due to small molecule-dependent heterodimerizing domains in each polypeptide of the CAR.

The cytoplasmic signaling domain (the intracellular signaling domain or the activating endodomain) of the CAR is responsible for activation of at least one of the normal effector functions of the immune cell in which the CAR is expressed, if the respective CAR is an activating CAR (normally, a CAR as described herein refers to an activating CAR, otherwise it is indicated explicitly as an inhibitory CAR (iCAR)). “Effector function” means a specialized function of a cell, e.g. in a T cell an effector function may be cytolytic activity or helper activity including the secretion of cytokines. The intracellular signaling domain refers to the part of a protein which transduces the effector function signal and directs the cell expressing the CAR to perform a specialized function. The intracellular signaling domain may include any complete, mutated or truncated part of the intracellular signaling domain of a given protein sufficient to transduce a signal which initiates or blocks immune cell effector functions.

Prominent examples of intracellular signaling domains for use in the CARs include the cytoplasmic signaling sequences of the T cell receptor (TCR) and co-receptors that initiate signal transduction following antigen receptor engagement.

Generally, T cell activation can be mediated by two distinct classes of cytoplasmic signaling sequences, firstly those that initiate antigen-dependent primary activation through the TCR (primary cytoplasmic signaling sequences, primary cytoplasmic signaling domain) and secondly those that act in an antigen-independent manner to provide a secondary or co-stimulatory signal (secondary cytoplasmic signaling sequences, co-stimulatory signaling domain). Therefore, an intracellular signaling domain of a CAR may comprise one or more primary cytoplasmic signaling domains and/or one or more secondary cytoplasmic signaling domains.

Primary cytoplasmic signaling domains that act in a stimulatory manner may contain ITAMs (immunoreceptor tyrosine-based activation motifs).

Examples of ITAM containing primary cytoplasmic signaling domains often used in CARs are that those derived from TCRζ (CD3ζ), FcRgamma, FcRbeta, CD3gamma, CD3delta, CD3epsilon, CD5, CD22, CD79a, CD79b, and CD66d. Most prominent is sequence derived from CD3ζ.

The cytoplasmic domain of the CAR may be designed to comprise the CD3ζ signaling domain by itself or combined with any other desired cytoplasmic domain(s). The cytoplasmic domain of the CAR can comprise a CD3ζ chain portion and a co-stimulatory signaling region (domain). The co-stimulatory signaling region refers to a part of the CAR comprising the intracellular domain of a co-stimulatory molecule. A co-stimulatory molecule is a cell surface molecule other than an antigen receptor or their ligands that is required for an efficient response of lymphocytes to an antigen. Examples for a co-stimulatory molecule are CD27, CD28, 4-1BB (CD137), OX40, CD30, CD40, PD-1, ICOS, lymphocyte function-associated antigen-1 (LFA-1), CD2, CD7, LIGHT, NKG2C, B7-H3.

The cytoplasmic signaling sequences within the cytoplasmic signaling part of the CAR may be linked to each other with or without a linker in a random or specified order. A short oligo- or polypeptide linker, which is preferably between 2 and 10 amino acids in length, may form the linkage. A prominent linker is the glycine-serine doublet.

As an example, the cytoplasmic domain may comprise the signaling domain of CD3ζ and the signaling domain of CD28. In another example the cytoplasmic domain may comprise the signaling domain of CD3ζ and the signaling domain of CD137. In a further example, the cytoplasmic domain may comprise the signaling domain of CD3ζ, the signaling domain of CD28, and the signaling domain of CD137.

As aforementioned either the extracellular part or the transmembrane domain or the cytoplasmic domain of a CAR may also comprise a heterodimerizing domain for the aim of splitting key signaling and antigen recognition modules of the CAR.

The CAR may be further modified to include on the level of the nucleic acid encoding the CAR one or more operative elements to eliminate CAR expressing immune cells by virtue of a suicide switch. The suicide switch can include, for example, an apoptosis inducing signaling cascade or a drug that induces cell death. In one embodiment, the nucleic acid expressing and encoding the CAR can be further modified to express an enzyme such thymidine kinase (TK) or cytosine deaminase (CD). The CAR may also be part of a gene expression system that allows controlled expression of the CAR in the immune cell. Such a gene expression system may be an inducible gene expression system and wherein when an induction agent is administered to a cell being transduced with said inducible gene expression system, the gene expression system is induced and said CAR is expressed on the surface of said transduced cell.

In some embodiments, the endodomain may contain a primary cytoplasmic signaling domains or a co-stimulatory region, but not both.

In some embodiment of the invention the CAR may be a “SUPRA” (split, universal, and programmable) CAR, where a “zipCAR” domain may link an intra-cellular costimulatory domain and an extracellular leucine zipper (WO2017/091546). This zipper may be targeted with a complementary zipper fused e.g. to an scFv region to render the SUPRA CAR T cell tumor specific. This approach would be particularly useful for generating universal CAR T cells for various tumors; adapter molecules could be designed for tumor specificity and would provide options for altering specificity post-adoptive transfer, key for situations of selection pressure and antigen escape.

If the CAR is an inhibitory CAR (referred to herein normally as “iCAR”) that may be expressed in addition to an activating CAR as described above in a cell, then said iCAR may have the same extracellular and/or transmembrane domains as the activating CAR but differs from the activating CAR with regard to the endodmain.

The at least one endodomain of the inhibitory CAR may be a cytoplasmic signaling domain comprising at least one signal transduction element that inhibits an immune cell or comprising at least one element that induces apoptosis.

Inhibitory endodomains of an iCAR are well-known in the art and have been described e.g. in WO2015075469A1, WO2015075470A1, WO2015142314A1, WO2016055551A1, WO2016097231A1, WO2016193696A1, WO2017058753A1, WO2017068361A1, WO2018061012A1, and WO2019162695A1.

The CARs of the present invention may be designed to comprise any portion or part of the above-mentioned domains as described herein in any order and/or combination resulting in a functional CAR, i.e. a CAR that mediated an immune effector response of the immune effector cell that expresses the CAR as disclosed herein.

The term “tagged polypeptide” as used herein refers to a polypeptide that has bound thereto directly or indirectly at least one additional component, i.e. the tag. The tagged polypeptide as used herein is able to bind an antigen expressed on a target cell. The polypeptide may be an antibody or antigen binding fragment thereof that binds to an antigen expressed on the surface of a target cell such as a tumor associated antigen on a cancer cell. The polypeptide of the tagged polypeptide alternatively may a cytokine or a growth factor or another soluble polypeptide that is capable of binding to an antigen of a target cell.

The terms “adapter” or “adapter molecule” or “tagged polypeptide” as used herein may be used interchangeably.

The tag may be e.g. a hapten or dextran and the hapten or dextran may be bound by the antigen binding domain of the polypeptide, e.g. a CAR, comprising an antigen binding domain specific for the tag.

Haptens such as e.g. FITC, biotin, PE, streptavidin or dextran are small molecules that elicit an immune response only when attached to a large carrier such as a protein; the carrier may be one that also does not elicit an immune response by itself. Once the body has generated antibodies to a hapten-carrier adduct, the small-molecule hapten may also be able to bind to the antibody, but it will usually not initiate an immune response; usually only the hapten-carrier adduct can do this.

But the tag may also be a peptide sequence e.g. chemically or recombinantly coupled to the polypeptide part of the tagged polypeptide. The peptide may be selected from the group consisting of c-Myc-tag, Strep-Tag, Flag-Tag, and Polyhistidine-tag. The tag may also be streptavidin. The tag portion of the tagged polypeptide is only constrained by being a molecular that can be recognized and specifically bound by the antigen binding domain specific for the tag of the CAR. For example, when the tag is FITC (Fluorescein isothiocyanate), the tag-binding domain may constitute an anti-FITC scFv. Alternatively, when the tag is biotin or PE (phycoerythrin), the tag-binding domain may constitute an anti-biotin scFv or an anti-PE scFv.

### EMBODIMENTS

In addition to above described applications of the process as disclosed herein, further embodiments of the invention are described in the following without intention to be limited to these embodiments.

In one embodiment of the invention the method comprises
a method for generating a composition of T cells expressing two exogenous TCRs under the control of the endogenous promoter of said T cell, wherein each single T cell of said composition expresses only one exogenous TCR, the method comprising
a) introducing into said T cells a first nucleic acid sequence encoding a TRBC specific TALENs, wherein said TALENs induces a cleavage at a specific cleavage site within the TRBC locus of the genome of said T cells, said cleavage resulting in an inhibition of expression of the endogenous TRBC gene,
b) introducing into said T cells two further nucleic acid sequence encoding an exogenous TCR, respectively, wherein each of said two further nucleic acid sequence comprises from 5′ to 3′:
a) a nucleic acid sequence homologous to the 5′ region upstream of said cleavage site (left homology arm, LHA)
b) a nucleic acid sequence encoding one TCR, and
c) a nucleic acid sequence homologous to the 3′ region downstream of said cleavage site (right homology arm, RHA),
thereby inserting said one TCR into said endogenous locus by homologous recombination, wherein either said LHA or said RHA is modified in such that the engineered nuclease (TRBC specific TALENs) is not able to bind in the nucleic acid sequences of LHA and RHA, thereby preventing binding of the engineered TALENs to both the integrated TCR as well as the non-integrated TCR due to loss of said specific cleavage site in said T cells and thereby inserting stably said one TCR into one endogenous TRBC locus driven under the control of an endogenous promotor in one T cell of the composition (the population), and thereby generating of two kinds of T cells within said composition (or population) of T cells expressing said two TCRs, respectively,
wherein said endogenous TRBC locus of said T cells underwent the process of allelic exclusion in said T immune cells, resulting in only one transcriptionally active and expressed allele of the originally gene located in said endogenous locus.

In another embodiment of the invention the method comprises a method for generating a composition of T cells expressing two exogenous TCRs under the control of the endogenous promoter of said T cell, wherein at least 80%, at least 85% or at least 90% of the immune cells of said composition express only one exogenous TCR, the method comprising
a) introducing into said T cells a first nucleic acid sequence encoding a TRBC specific TALENs, wherein said TALENs induces a cleavage at a specific cleavage site within the TRBC locus of the genome of said T cells, said cleavage resulting in an inhibition of expression of the endogenous TRBC gene,
b) introducing into said T cells two further nucleic acid sequence encoding an exogenous TCR, respectively, wherein each of said two further nucleic acid sequence comprises from 5 to 3:
a) a nucleic acid sequence homologous to the 5′ region upstream of said cleavage site (left homology arm, LHA)
b) a nucleic acid sequence encoding one TCR, and
c) a nucleic acid sequence homologous to the 3′ region downstream of said cleavage site (right homology arm, RHA),
thereby inserting said one TCR into said endogenous locus by homologous recombination, wherein either said LHA or said RHA is modified in such that the engineered nuclease (TRBC specific TALENs) is not able to bind in the nucleic acid sequences of LHA and RHA, thereby preventing binding of the engineered TALENs to both the integrated TCR as well as the non-integrated TCR due to loss of said specific cleavage site in said T cells and thereby inserting stably said one TCR into one endogenous TRBC locus driven under the control of an endogenous promotor in one T cell of the composition (the population), and thereby generating at least 80%, at least 85% or at least 90% of T cells within said composition (or population) that express only one of said two TCRs in a single cell.

In another embodiment of the invention T cells may be co-electroporated with TALENs as well as a nucleic acid sequence encoding the exogenous TCRs delivered as linearized DNA.

In another embodiment of the invention T cells may be co-electroporated with TALENs as well as a nucleic acid sequence encoding the exogenous TCRs delivered as plasmid.

In another embodiment of the invention T cells may be electroporated with TALENs and subsequently transduced with adenovirus-associated virus 6 (AAV6)-derived vectors encoding the exogenous TCRs.

In another embodiment of the invention TALENs are provided either as mRNA or DNA and delivered via electroporation into the T cell.

In another embodiment of the invention TALENs are transduced into T cells using, but not limited to, lentiviral vectors, retroviral vectors or AAV6.

In another embodiment of the invention the TCRs may be flanked with up to two different left homology arms and up to two different right homology arms as disclosed herein.

In another embodiment of this invention flanking homology arms are not longer than 200, not longer than 300, not longer than 400, not longer than 500 nucleotides, not longer than 600, not longer than 700, not longer than 800, not longer than 900, not longer than 1000, not longer than 1100, not longer than 1200 nucleotides, not longer than 1300, not longer than 1400, or not longer than 1500 nucleotides respectively.

In another embodiment on the invention a plurality of further nucleic acid sequences encoding a plurality of exogenous TCRs are delivered into said T cells as linearized DNA, plasmid DNA, AAV6 or combinations thereof.

In another embodiment of the invention the method comprises
a method for generating a composition of T cells expressing two exogenous TCRs under the control of the endogenous promoter of said T cell, wherein each single T cell of said composition expresses only one exogenous TCR, the method comprising
a) introducing into said T cells a first nucleic acid sequence encoding a TRBC specific clustered, regularly interspaced, short palindromic repeats (CRISPR)-associated Cas9 protein, wherein said CRISPR/Cas9 induces a cleavage at a specific cleavage site within the TRBC locus of the genome of said T cells, said cleavage resulting in an inhibition of expression of the endogenous TRBC gene,
b) introducing into said T cells two further nucleic acid sequence encoding an exogenous TCR, respectively, wherein each of said two further nucleic acid sequence comprises from 5 to 3:
a) a nucleic acid sequence homologous to the 5′ region upstream of said cleavage site (left homology arm, LHA)
b) a nucleic acid sequence encoding one TCR, and
c) a nucleic acid sequence homologous to the 3′ region downstream of said cleavage site (right homology arm, RHA),
thereby inserting said one TCR into said endogenous locus by homologous recombination, wherein either said LHA or said RHA is modified in such that the engineered nuclease (TRBC specific CRISPR/Cas9) is not able to bind in the nucleic acid sequences of LHA and RHA, thereby preventing binding of the engineered nuclease (CRISPR/Cas9) to both the integrated TCR as well as the non-integrated TCR due to loss of said specific cleavage site in said T cells and thereby inserting stably said one TCR into one endogenous TRBC locus driven under the control of an endogenous promotor in one T cell of the composition (the population), and thereby generating of two kinds of T cells within said composition (or population) of T cells expressing said two TCRs, respectively,
wherein said endogenous TRBC locus of said T cells underwent the process of allelic exclusion in said T immune cells, resulting in only one transcriptionally active and expressed allele of the originally gene located in said endogenous locus.

As described in the embodiments above, the further nucleic acid sequences encoding a exogenous TCRs may be delivered as linearized DNA, plasmid DNA, AAV6 or combinations thereof.

In another embodiment of the invention the exogenous TCRs may belong but are not limited to the group of neo-antigen specific TCRs or shared tumor-associated antigens. More specifically, potential TCR therapy targets may include but are not limited to Melanoma-associated antigen (MAGE)-A1, MAGE-A2, MAGE-A3, MAGE-A4, MAGE-A6, MAGE-A10, and MAGE-A12, glycoprotein (gp100), melanoma antigen recognized by T cells (MART-1), tyronsinase, carcinoembryonic antigen (CEA), prostate-specific antigen (PSA), Wilms tumor 1 (WT1), dNPM1, Mesothelin, NY-ESO-1, PRAME, p53, HPV-E6, TRAIL, DR4, Thyroglobulin, TGFβII frameshift antigen, LAGE-1A, KRAS G12V, HPV-E7, HERV-E, HA-1, CMV, AFP and/or combinations thereof.

In another embodiment of the invention the exogenous TCRs specifically recognize antigens presented on, but not limited to, HLA A*0101, HLA A*0201, HLA A*0301, HLA A*1101, HLA A*2402, HLA A*2601, HLA A*2902, HLA A*3303, HLA A*6801, HLA B*0702, HLA B*0801, HLA B*1402, HLA B*1501, HLA B*1502, HLA B*1801, HLA B*2705, HLA B*3501, HLA B*4001, HLA B*4002, HLA B*4402**, HLA B*4403, HLA B*4501, HLA B*0702, HLA B*3501, HLA C*0102, HLA C*0202, HLA C*0303, HLA C*0304, HLA C*0401, HLA C*0501, HLA C*0602, HLA C*0701, HLA C*0702, HLA C*0801, HLA DRB1*0101, HLA DRB1*0301, HLA DRB1*0401, HLA DRB1*0701, HLA DRB1*0901, HLA DRB1*1501, HLA DRB1*1101, HLA DRB1*1301, HLA DRB1*1401, HLA DRB1*0404, HLA DRB1*0405, HLA DRB1*0407, HLA DRB1*0411, HLA DRB1*0803 and HLA DRB1*1302.

In another embodiment of the invention the nucleic acid sequences as described in the embodiments described above may encode CARs instead of exogenous TCRs. Then the CARs, after knockin into the TRCB locus, are expressed under the control of the endogenous promotor in genetically engineered T cells.

In another embodiment of the invention the CAR may be specific for following antigens but are not limited to: TNFRSF17, IL3RA, SDC1, EGFRVIII, MUC1, FAP, CD44, CD19, AS4A1, CD22, EPCAM, PDCD1, CA9, CD174, TNFRSF8, CD33, CD3δ, EPHA2, CD274, FOLR1, SLAMF7, CD5, NCAM1, CD70, ERBB2, KDR, LICAM, GD2, ULBP1, ULBP2, IL1RAP, GPC3, IL13RA2, ROR1, CEACAM5, MET, EGFR, MSLN, FOLH1, CD23, CD276, CSPG4, CD133, TEM1, GPNMB, PSCA, FLT-3, CD20, HER2, CD227, CLDN18.2, FOLR1, BCMA, Biotin, CD123, CD138, CS1, Mesothelin, MUC1, MUC16, Nectin4, Glypican 3, B7H3, GPC3 and/or combinations thereof.

In another embodiment of the invention a plurality of CARs and TCRs are used, wherein a plurality of further nucleic acid sequences encoding a plurality of CARs and TCRs, and wherein said method is for generating a population of T cells expressing a plurality of CARs and TCRs under the control of the endogenous promoter of said T cells, respectively, wherein each single T cell of said composition expresses only one CAR or TCR.

In another embodiment of the invention one CAR and one TCR are used, wherein two further nucleic acid sequences encoding said CAR and TCR, respectively, and wherein said method is for generating a population of T cells expressing said CAR and said TCR under the control of the endogenous promoter of said T cells, respectively, wherein each single T cell of said composition expresses only the CAR or the TCR.

In another embodiment of the invention a meganuclease, s zinc-finger nuclease (ZFN) a transcription activator-like effector nuclease (TALE-Nuclease), a CRISPR/Cas nuclease, MAD7 nuclease, CRISPR/Cpf1, Cas12-type-derived nucleases or a megaTAL nuclease, as mentioned in an embodiment above, can be either provided as mRNA or DNA and delivered via electroporation into the T cell or transduced into T cells using, but not limited to, lentiviral vectors, retroviral vectors or AAV6.

In another embodiment of the invention an automated and closed system such as, but not limited to, the CliniMACS Prodigy may be used to manufacture the genetically engineered population of immune cells such as T cells.

### EXAMPLES

The following examples are intended for a more detailed explanation of the invention but without restricting the invention to these examples.

### Example 1. Genetic Modification of T Cells Combining Knockout and Targeted Knockin

1.1 gRNA and Donor DNA Template Design

The TRBC gene sequences including TRBC1 and TRCB2 are retrieved from NCBI database. A gRNA is designed to target Exon 1 of the TRBC locus (SEQ ID NO: 1) covering both genes. For the electroporation, Cas9 ribonucleoproteins (RNPs) are produced and subsequently used for electroporation. Homology directed repair is induced using P2A-linked (SEQ ID NO: 2) exogenous TCR encoding sequences flanked with one 5′ and one or two 3′ homology arms (HA) (SEQ ID NO: 3, SEQ ID NO: 4 & SEQ ID NO: 5), respectively. When using DNA templates, gRNA targeting sites as well as protospacer adjacent motif (PAM) sequences are added 5′ and 3′ allowing to linearize the circular plasmid in vivo using the same endonuclease as used for the endogenous ko. Linearization in vitro is possible using EcoRI 5′ and 3′ flanking the HA. Donor DNA templates are co-electroporated. Alternatively, AAV6 can be used to deliver the donor template for the targeted knockin. Therefore, cells are transduced 2-4 h post electroporation. In both cases, if a TCR is used, an additional cutting of the CRISPR/Cas9 tool has to be prevented. Then, the PAM sequence in the TRBC locus encoding sequence in the exogenous TCR is deleted by changing the wobble base.

### 1.2 TALENs and Alternative Donor DNA Template Design

Alternatively, the knockout can be induced using TALENs. Therefore, a TALEN pair is designed to cleave the first exon of the of the TRBC gene. TALEN encoding plasmids including a T7 promoter are linearized and used as templates for mRNA production by in vitro transcription (IVT) using T7 RNA polymerase. The in vitro mRNA transcripts are purified using RNeasy kit (Qiagen), enzymatically capped, and polyadenylated. After DNase treatment, RNA concentrations are determined by measuring the absorbance at 260 nm using a Nanodrop spectrophotometer (ThermoFisher). The length of the in vitro transcripts and the polyadenylated mRNAs are monitored by Bioanalyzer electrophoreses on an RNA Nano chip (Agilent). Donor DNA templates are designed as already described, however, containing alternative combinations of 5′ and 3′ HA. If a TCR is used, an additional cutting of the TALENs has to be prevented. Therefore, the binding sequence of the left TALEN in the exogenous TCR is deleted by using an alternative codon usage.

### 1.3 Automated T Cell Enrichment, Engineering and Expansion

Unless mentioned to the contrary, kits and reagents are used according to the manufacturer's protocol. All kits and reagents, unless mentioned otherwise, are from Miltenyi Biotec. TCR-engineered T cells are manufactured using an automated and closed T Cell Engineering (TCE) Process on the CliniMACS Prodigy® platform during the entire process. Blood products are analyzed for total WBC concentration and target cell frequency (CD4+ plus CD8+ among CD45+) prior to process and a maximum of 3×10⁹ target cells is processed on the CliniMACS Prodigy. Process buffer CliniMACS PBS/EDTA supplemented with 0.5% BSA or 0.5% HSA (Grifols) as well as TexMACS GMP Medium supplemented with 3% heat-inactivated human AB Serum (Gemini Bio-Products), 12.5 ng/mL recombinant human IL-7 and 12.5 ng/mL recombinant human IL-15 are prepared before starting the automated process. Process parameters are entered into the “activity matrix” in order to define the conditions of the automated run. Initial priming, sample preparation and cell labeling is performed by the instrument at 4-8° C. Cell labeling with magnetic beads is performed using CliniMACS CD4 Reagent and CliniMACS CD8 Reagent for 30 min at 4-8° C. After magnetic separation, target cells are eluted in TexMACS GMP medium and cell concentration of the enriched fraction is determined by the operator. For the automated cultivation, a maximum of 2×10⁸ T cells are seeded in the chamber of the CliniMACS Prodigy, washed and T cells are activated with 1 vial of the MACS GMP T Cell TransAct. At day 3 post-activation, cells are re-buffered in CliniMACS® Electroporation Buffer and subsequently co-electroporated with either RNPs according to the manufacturer's protocol (Integrated DNA Technologies) or 80 μg-120 μg TRBC specific mRNA encoding TALENs as well as 5-10 μg of up to 6 different donor DNA templates (either pre-linearized using EcoRI or circular) per shot, respectively. Following electroporation, cells are transferred back to the cultivation chamber and recovered in 66 ml TexMACS media supplemented with IL-7, IL-15 as well as 3% AB serum and static culture is performed for 24 hours and then switched back to agitated modus. Alternatively, if using AAV6 as donor DNA delivery system, cells were transduced 2-4 h post electroporation. Afterwards, cells are expanded for up to 14 days. Medium is changed automatically via centrifugation and automatic media feed every other day. Cultivation samples are taken frequently. Cell count as well as viability is analyzed via flow cytometry. For the enriched fraction (day 5), the in-process control (day 5/6) and for the final cellular product (post-harvest) samples are taken for flow cytometric analysis to determine cellular composition, T cell phenotype and transduction efficiency.

### 1.4 Flow Cytometric Analysis

The flow cytometric analysis reveals that all transgenes, encoded by the donor DNA templates, used during the electroporation are present in the cells of the composition in equal ratios.

1.5 gRNA and Alternative Donor DNA Delivery with Adeno-Associated-Virus 6 (AAV6)

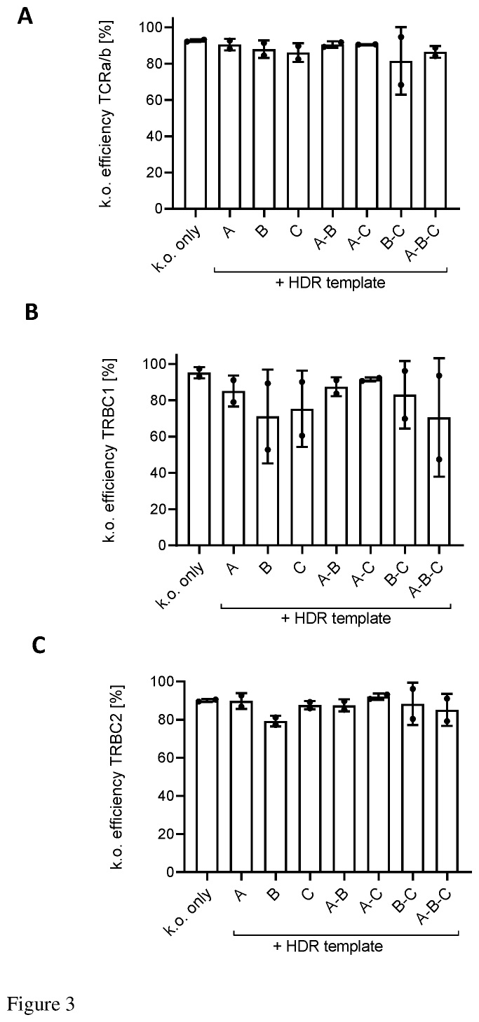

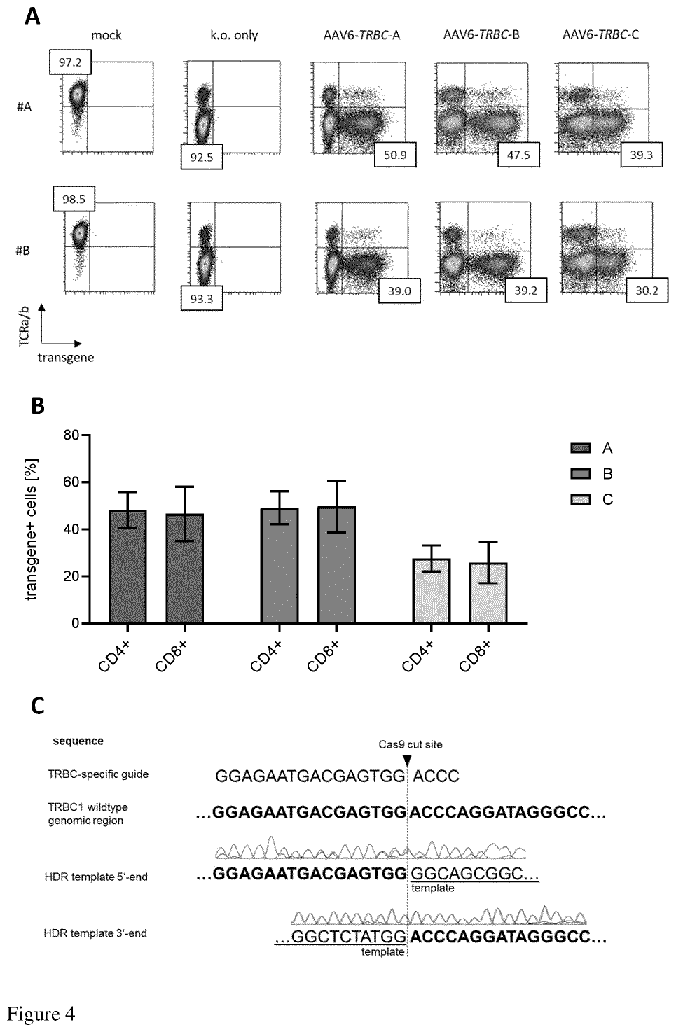

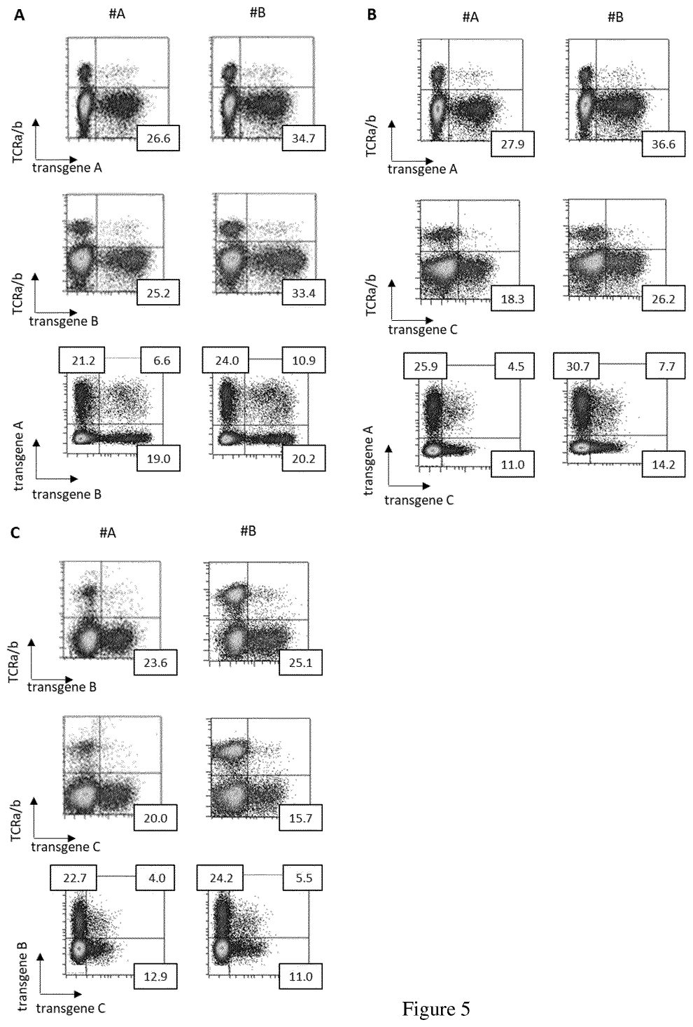

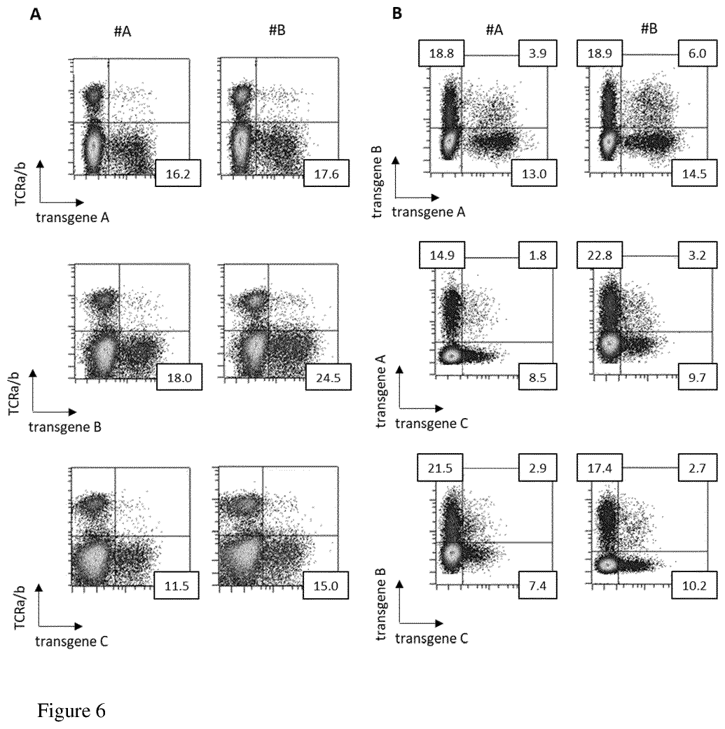

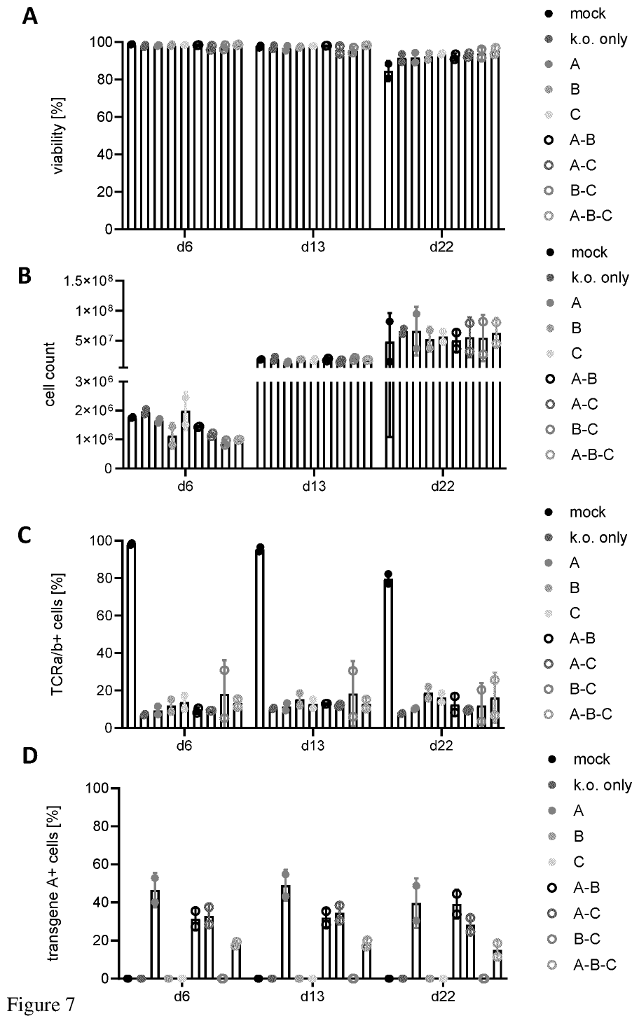

Transduction of T cells with HDR template containing AAV6 vectors is one method to deliver the donor template for a targeted knockin into specific loci. Unless mentioned to the contrary, kits and reagents are used according to the manufacturer's protocol. All kits and reagents, unless mentioned otherwise, are from Miltenyi Biotec. For this approach, activated T cells are transduced 1-1.5 h pre-electroporation at day 3 with AAV6 vectors each containing a TRBC-targeted HDR template coding for a specific transgene (AAV6-TRBC-A, AAV6-TRBC-B, AAV6-TRBC-C). Homology directed repair is induced using P2A-linked (SEQ ID NO: 2) transgene encoding sequences flanked with one 5′ and one or two 3′ homology arms (HA) (SEQ ID NO: 3, SEQ ID NO: 4 & SEQ ID NO: 5), respectively. Transgene amino acid sequences were retrieved from UniProt database: transgene A (GFP, SEQ ID NO: 6), B (delta-LNGFR, SEQ ID NO: 7) or C (CD20, SEQ ID NO: 8). T cells are either single-transduced with HDR donor template containing AAV6 (AAV6-TRBC-A, AAV6-TRBC-B or AAV6-TRBC-C) or double- or triple-transduced with different combinations of AAV6-TRBC-A and AAV6-TRBC-B, AAV6-TRBC-A and AAV6-TRBC-C, AAV6-TRBC-B and AAV6-TRBC-C or AAV6-TRBC-A and AAV6-TRBC-B and AAV6-TRBC-C using an MOI of 3E4 for each vector. For the specific double-strand break at the knockin site, TRBC-specific Cas9 ribonucleoproteins (RNPs) are used and inserted into the transduced T cells via electroporation. RNP production is performed by complexing 300 pmol of guide RNA with 100 pmol Cas9 protein for 20 min at RT. The RNPs are then mixed with 1 E6 AAV6-transduced T cells in a total volume of 50 μl electroporation buffer and electroporated with the CliniMACs electroporator unit of the CliniMACS Prodigy using the following bi-pulse: 950 V, 104 μs burst/bipolar; 400 V 2 ms burst. Viability, cell count, knockout efficiency and knockin efficiency is assessed at d6, d13 and d22 via co-staining of 7-AAD, α-LNGFR-antibody, α-CD20-antibody and α-TCRa/b-antibody and GFP measurement using flow cytometry. For the estimation of knockout efficiency cells are either stained with α-TCRa/b antibody or co-stained with α-TCRa/b-antibody and α-TRBC1-antibody (ThermoFischer), where single TCRa/b positive cells are considered as TCRBC2 T cells and double positive cells as TRBC1 T cells. TRBC-specific Cas9 RNP electroporation induces an efficient knockout and loss of TCRa/b (mean: 92.47%), as well as TRBC1 (mean: 99.79%) and TRRBC2 (mean: 96.96%) expression in RNP-only electroporated cells and equally in all combinations of AAV6-transduced T cells (FIG. 3). After AAV6-mediated HDR delivery and TRBC-specific Cas9 RNP electroporation, high targeted knockin efficiencies into the TRBC locus of T cells are achieved for all transgenes (A: mean: 40.07%, B: mean: 44.42%, C: mean: 34.3%) when single-transduced with an AAV6 containing donor template (FIG. 4). Seamless integration into the specific genomic region of TRBC is confirmed trough Sanger-Sequencing of Polymerase-Chain-Reaction amplified TRBC1 or TRBC2 DNA sequences using 3′ and 5′ outward transgene-binding primers (FIG. 4). Dual HDR delivery results in efficient knockin of transgenes and a T cell population in which two transgenes are expressed and wherein each transgene is evenly distributed (f.e. 26.6% and 25.2%) (FIG. 5). Targeted and efficient transgene knockin and similar ratios of transgene expression (f.e. 16.2%, 18% and 11.5%) are also observed in AAV6 triple-transduced T cells (FIG. 6). Within the T cell population used for multiplex gene-editing, a small proportion of T cells with deficient allelic exclusion and double transgene expression (up to 11%) was identified (FIGS. 5 and 6). Genetically-engineered T cells show high viability, cellular expansion and stable knockout efficiency as well as stable knockin efficiency of transgenes in all conditions over time (FIG. 7).

### REFERENCES

(1) Mock, U, Nickolay, L, Philip, B, et al., Automated manufacturing of chimeric antigen receptor T cells for adoptive immunotherapy using CliniMACS prodigy. Cytotherapy, 2016
(2) Lock D, Mockel-Tenbrinck N, Drechsel K, Barth C, Mauer D, Schaser T, Kolbe C, Al Rawashdeh W, Brauner J, Hardt O, Pflug N, Holtick U, Borchmann P, Assenmacher M, Kaiser A. Automated manufacturing of potent CD20-directed CAR T cells for clinical use. Hum Gene Ther. 2017
(3) Priesner, C, Aleksandrova, K, Esser, R, et al., Automated enrichment, transduction and expansion of clinical-scale CD62L+ T cells for manufacturing of GTMPs. Hum Gene Ther, 2016
(4) Alzubi J, Lock D, Rhiel M, Schmitz S, Wild S, Mussolino C, Hildenbeutel M, Brandes C, Rositzka J, Lennartz S, Haas S A, Chmielewski K O, Schaser T, Kaiser A, Cathomen T, Cornu T I. Automated generation of gene-edited CAR T cells at clinical scale. Mol Ther Methods Clin Dev. 2020

1-15. (canceled)

16. A method for generating a composition of immune cells that express a plurality of transgenes under control of an endogenous promoter of an endogenous locus of said immune cells,
wherein each single immune cell of said composition that underwent allelic exclusion with regard to said endogenous locus expresses only one transgene,
wherein the transgene encodes a therapeutic protein or a therapeutic nucleic acid,
wherein the method comprises:
(i) introducing into primary immune cells a first nucleic acid sequence encoding an engineered nuclease, wherein said engineered nuclease induces cleavage at a specific cleavage site within an endogenous locus of the genome of said immune cell, said cleavage resulting in an inhibition of expression of the endogenous gene of said endogenous locus,
(ii) introducing into said primary immune cells a plurality of further nucleic acid sequences encoding a plurality of transgenes, wherein each individual further nucleic acid sequence of said plurality of further nucleic acid sequences comprises from 5′ to 3′:
a) a nucleic acid sequence homologous to the 5′ region upstream of said cleavage site (left homology arm, LHA),
b) a nucleic acid sequence encoding one transgene, and
c) a nucleic acid sequence homologous to the 3′ region downstream of said cleavage site (right homology arm, RHA), thereby inserting said one transgene into said endogenous locus by homologous recombination,
wherein either said LHA or said RHA is modified such that the engineered nuclease is not able to bind in the nucleic acid sequences of LHA and RHA, thereby preventing binding of the engineered nuclease to both the integrated transgene as well as the non-integrated transgene due to loss of said specific cleavage site in said immune cells, thereby inserting stably said one transgene into one endogenous locus driven under the control of an endogenous promotor in one immune cell of the composition, thereby generating a plurality of immune cells within said composition of immune cells expressing the plurality of transgenes,
wherein the plurality of transgenes comprises at least two different transgenes, and
wherein said immune cells are T cells.

17. The method according to claim 16, wherein said engineered nuclease is a meganuclease, a zinc finger nuclease (ZFN) a transcription activator-like effector nuclease (TALE-Nuclease), a CRISPR/Cas nuclease, MAD7 nuclease, CRISPR/Cpf1, Cas12-type-derived nucleases or a megaTAL nuclease.

18. The method according to claim 16, wherein said endogenous locus of said immune cells is selected from the group consisting of T cell receptor beta locus, T cell receptor gamma locus, and T cell receptor delta locus.

19. The method according to claim 16, wherein said endogenous locos of said immune cells is the T cell receptor beta locus, which comprises either the T cell receptor beta 1 constant (TRBC1) gene or the T cell receptor beta 2 constant (TRBC2) gene, and said immune cells are T cells.

20. The method according to claim 19, wherein said specific cleavage site is in exon 1 of the T cell receptor beta 1 constant gene or in exon 1 of the T cell receptor beta 2 constant gene,
wherein said engineered nuclease induces said cleavage at said specific cleavage site in exon 1 of the T cell receptor beta I constant gene and in exon 1 of the T cell receptor beta 2 constant gene, dependent on which T cell receptor beta constant gene is present after T cell receptor gene rearrangement during thymocyte development in said immune cell as the sequence at which is cleaved is identical in both exons I of the T cell receptor beta constant genes,
wherein said each individual further nucleic acid sequence of said plurality of further nucleic acid sequences comprises downstream of said RHA (RHA1) a second RHA (RHA2),
wherein said RHA1 has a nucleic acid sequence homologous to the 3′ region downstream of said cleavage site in exon 1 of the T cell receptor beta 1 constant gone,
wherein said RHA2 has a nucleic acid sequence homologous to the 3′ region downstream of said cleavage site in exon 1 of the T cell receptor beta 2 constant gene, or vice versa, and
wherein said nucleic acid sequences of RHA 1 and RHA2 are different,
thereby allowing homologous recombination in said locos in said immune cells independently therefrom if the T cell receptor beta 1 constant gene or T cell receptor beta 2 constant gene has been rearranged in a single immune cell of said composition.

21. The method according to claim 20, wherein said specific cleavage site is in exon 2 of the T cell receptor beta 1 constant gene or in exon 2 of the T cell receptor beta 2 constant gene,
wherein said engineered nuclease induces said cleavage at said specific cleavage site in exon 2 of the T cell receptor beta 1 constant gene and in exon 2 of the T cell receptor beta 2 constant gene, dependent on which T cell receptor beta constant gene is present after T cell receptor gene rearrangement during thymocyte development in said immune cell as the sequence at which is cleaved is identical in both exons 2 of the T cell receptor beta constant genes,
wherein said each individual further nucleic acid sequence of said plurality of further nucleic acid sequences comprises upstream of said LHA (LHA1) a second LHA (LHA2),
wherein said LHA1, has a nucleic acid sequence homologous to the 5′ region upstream of said cleavage site of the T cell receptor beta I constant gene, and
wherein said LHA2 has a nucleic acid sequence homologous to the 5′ region upstream of said cleavage site of the T cell receptor beta 2 constant gene, or vice versa,
wherein said nucleic acid sequences of LHA1 and LHA2 are different,
thereby allowing homologous recombination in said locus in said immune cells independently therefrom if the T cell receptor beta 1 constant gene or T cell receptor beta 2 constant gene has been rearranged in a single immune cell of said composition.

22. The method according to claim 16, wherein the plurality of transgenes are exogenous T cell receptors (TCRs) and/or chimeric antigen receptors (CARs).

23. The method according to according to claim 16, wherein the plurality of transgenes are exogenous T cell receptors (TCRs), and
wherein said nucleic acid sequence encoding said one transgene of said each individual further nucleic acid sequence of said plurality of further nucleic acid sequences comprises the T cell receptor alpha chain and the T cell receptor beta chain comprising a variable and a constant domain, respectively.

24. The method according to according to claim 16, wherein said at least one further nucleic acid sequence is a plasmid, a linearized plasmid, or a viral vector.

25. The method according to claim 24, wherein said viral vector is adeno-associated virus vector such as adeno-associated virus type 6 (AAV6) vector.

26. The method according to claim 16, wherein said immune cells of step (i) and step (ii) are activated T cells, and wherein said method comprises before step (i) and before step (ii):
A) preparing said primary immune cells, wherein said primary immune cells are T cells,
B) separating said T cells by magnetic separation, and
C) activating the enriched T cells using modulatory agents.

27. The method according to claim 26, wherein in step (i) and step (i) the introducing is performed by electroporation or transduction.

28. The method according to claim 26, further comprising after step (1) and step (ii):
D) expanding the genetically modified T cells.

29. The method according to claim 26, wherein said method is performed in a closed system.

30. The method according to claim 26, wherein said primary T cells comprise 2×10⁸ primary T cells.

31. A composition of immune cells generated according to the method of claim 16,
wherein the immune cells express a plurality of transgenes under the control of an endogenous promoter of an endogenous locus of said immune cells,
wherein each single immune cell of said composition expresses only one transgene,
wherein the one transgene encodes a therapeutic protein or therapeutic nucleic acid, and
wherein the plurality of transgenes in the composition comprises at least two different transgenes.

32. The composition according to claim 26, compounded with a pharmaceutically acceptable carrier, diluent or excipient for use in human therapy.
